(19) United States
(12) Patent Application Publication
(10) Pub. No.: US 2024/0270833 A1
(43) Pub. Date: Aug. 15, 2024

(54) Means and Methods for the Determination of the Biological Activity of Neurotoxin Polypeptides in Cells

(71) Applicant: MERZ PHARMA GmbH & CO. KGaA, FRANKFURT am MAIN (DE)

(72) Inventor: Cornelia BRÜNN, Frankfurt am Main (DE)

(21) Appl. No.: 18/621,418

(22) Filed: Mar. 29, 2024

Related U.S. Application Data
(62) Division of application No. 14/901,123, filed on Dec. 28, 2015, now Pat. No. 11,976,110.

(30) Foreign Application Priority Data
Jun. 28, 2013 (EP) 13174176.4

(51) Int. Cl. C07K 16/18; G01N 21/64; G01N 33/50

(52) U.S. Cl. CPC C07K 16/18; G01N 21/6428; G01N 33/5014; G01N 33/5058; G01N 33/5073; C07K 2317/34; G01N 2021/6441; G01N 2333/33; G01N 2333/952

(57) ABSTRACT

The invention pertains to a method for directly determining the biological activity of a Neurotoxin polypeptide in cells, comprising the steps of: a) incubating cells susceptible to Neurotoxin intoxication with a Neurotoxin polypeptide for a time and under conditions which allow for the Neurotoxin polypeptide to exert its biological activity; b) fixing the cells and, optionally, permeabilizing the cells with a detergent; c) contacting the cells with at least a first capture antibody specifically binding to the non-cleaved and Neurotoxin-cleaved substrate and with at least a second capture antibody specifically binding to the cleavage site of the Neurotoxin-cleaved substrate, under conditions which allow for binding of the capture antibodies to the substrates; d) contacting the cells with at least a first detection antibody specifically binding to the first capture antibody, under conditions which allow for binding of the first detection antibody to the first capture antibody, thus forming first detection complexes, and at least a second detection antibody specifically binding to the second capture antibody, under conditions which allow for binding of the second detection antibody to the second capture antibody, thus forming second detection complexes; e) determining the amount of the first and second detection complexes of step d), and f) calculating the amount of substrate cleaved by the Neurotoxin polypeptide in the cells by means of the second detection complexes, thereby determining the biological activity of the Neurotoxin polypeptide in the cells. The invention further provides for a kit for carrying out the method of the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 111,572 Bytes file named “MERZ_210_SEQ_US_DIV_SEQUENCE_LISTING.XML,” created on 22 Mar. 2024.

The present invention pertains to a method for directly determining the biological activity of a Neurotoxin polypeptide in cells, comprising the steps of: a) incubating cells susceptible to Neurotoxin intoxication with a Neurotoxin polypeptide for a time and under conditions which allow for the Neurotoxin polypeptide to exert its biological activity; b) fixing the cells and, optionally, permeabilizing the cells with a detergent; c) contacting the cells with at least a first capture antibody specifically binding to the non-cleaved and Neurotoxin-cleaved substrate and with at least a second capture antibody specifically binding to the cleavage site of the Neurotoxin-cleaved substrate, under conditions which allow for binding of said capture antibodies to said substrates; d) contacting the cells with at least a first detection antibody specifically binding to the first capture antibody, under conditions which allow for binding of said first detection antibody to said first capture antibody, thus forming first detection complexes, and at least a second detection antibody specifically binding to the second capture antibody, under conditions which allow for binding of said second detection antibody to said second capture antibody, thus forming second detection complexes; e) determining the amount of the first and second detection complexes of step d), and f) calculating the amount of substrate cleaved by said Neurotoxin polypeptide in said cells by means of the second detection complexes, thereby determining the biological activity of said Neurotoxin polypeptide in said cells. The invention further provides for a kit for carrying out the method of the invention.

Clostridium botulinum and Clostridium tetani produce highly potent Neurotoxins, i.e. Botulinum toxins (BoNTs) and Tetanus toxin (TeNT), respectively. These Clostridial Neurotoxins (CNTs) specifically bind to neuronal cells and disrupt neurotransmitter release. Each toxin is synthesized as an inactive unprocessed approximately 150 kDa single-chain protein. The posttranslational processing involves formation of disulfide bridges, and limited proteolysis (nicking) by the bacterial protease(s). Active Neurotoxin consists of two chains, an N-terminal light chain of approx. 50 kDa and a heavy chain of approx. 100 kDa linked by a disulfide bond. CNTs structurally and functionally consist of three domains, i.e. the catalytic light chain, the heavy chain encompassing the translocation domain (N-terminal half) and the receptor binding domain (C-terminal half); see, e.g., Krieglstein 1990, Eur. J. Biochem. 188, 39; Krieglstein 1991, Eur. J. Biochem. 202, 41; Krieglstein 1994, J. Protein Chem. 13, 49. The Botulinum Neurotoxins are synthesized as molecular complexes comprising the 150 kDa Neurotoxin protein and associated non-toxic proteins. The complex sizes differ based on the Clostridial strain and the distinct Neurotoxin serotypes ranging from 300 kDa, over 500 kDa, and 900 kDa. The non-toxic proteins in these complexes stabilize the Neurotoxin and protect it against degradation; see Silberstein 2004, Pain Practice 4, S19-S26.

Clostridium botulinum secretes seven antigenically distinct serotypes designated A to G of the Botulinum Neurotoxin (BoNT). All serotypes together with the related Tetanus Neurotoxin (TeNT) secreted by Clostridium tetani, are Zn²⁺-endoproteases that block synaptic exocytosis by cleaving SNARE proteins; see Couesnon, 2006, Microbiology, 152, 759. CNTs cause the flaccid muscular paralysis seen in botulism and tetanus; see Fischer 2007, PNAS 104, 10447.

Despite its toxic effects, Botulinum toxin complex has been used as a therapeutic agent in a large number of diseases. Botulinum toxin serotype A was approved for human use in the United States in 1989 for the treatment of strabism, blepharospasm, and other disorders. It is commercially available as Botulinum toxin A (BoNT/A) protein preparation, for example, under the trade name BOTOX (Allergan, Inc.) or under the trade name DYSPORT/RELOXIN (Ipsen, Ltd). An improved, complex-free Botulinum toxin A preparation is commercially available under the trade name XEOMIN (Merz Pharmaceuticals, LLC). For therapeutic applications, the preparation is injected directly into the muscle to be treated. At physiological pH, the toxin is released from the protein complex and the desired pharmacological effect takes place. The effect of Botulinum toxin is only temporary, which is the reason why repeated administration of Botulinum toxin may be required to maintain a therapeutic affect.

The Clostridial Neurotoxins weaken voluntary muscle strength and are effective therapy for strabism, focal dystonia, including cervical dystonia, and benign essential blepharospasm. They have been further shown to relief hemifacial spasm, and focal spasticity, and moreover, to be effective in a wide range of other indications, such as gastrointestinal disorders, hyperhidrosis, and cosmetic wrinkle correction; see Jost 2007, Drugs 67, 669.

During the manufacturing process of Clostridial Neurotoxins, the qualitative and quantitative determination of said Neurotoxins as well as the quality control of the biologically active Neurotoxin polypeptides is of particular importance. In addition, governmental agencies accept only simple, reliable, and validated Botulinum toxin activity assays. At present the mouse LD₅₀ bioassay, a lethality test, remains the “gold standard” used by pharmaceutical manufacturers to analyze the potency of their preparations; see Arnon et al. (2001), JAMA 285, 1059-1070. However, in recent years, considerable effort has been undertaken to seek for alternative approaches to alleviate the need for animal testing and all the disadvantages, costs and ethical concerns associated with this type of animal-based assays. In addition, the regulatory agencies are engaging pharmaceutical companies to apply the three “Rs” principle to the potency testing of Botulinum Neurotoxins: “Reduce, Refine, Replace”: see Straughan, Altern. Lab. Anim. (2006), 34, 305-313. As a consequence, cell-based test systems have been developed in order to provide reasonable alternatives to methods using live animals. Yet, only three cellular test systems are available for the determination of Neurotoxin biological activity thus far which have been shown to be sufficiently sensitive to Neurotoxin polypeptides. These cell-based test systems include the use of primary neurons isolated from rodent embryos which are differentiated in vitro (Pellett et al. (2011), Biochem. Biophys. Res. Commun. 404, 388-392), neuronal differentiated induced pluripotent stem cells (Whitemarsh et al. (2012), Toxicol. Sci. 126, 426-35), and a subclone of the SiMa cell line (WO 2010/105234 A1).

However, the isolation of primary neurons requires the killing of animals and is laborious and time consuming. Further, test systems using different primary neurons show large variances. Similarly, the generation of neuronal differentiated induced pluripotent stem cells is difficult and time consuming. In addition, storage of such cells is very problematic. Assays using tumor cell lines are frequently not sensitive enough to BoNT. Moreover, complex differentiation protocols are required for said tumor cell lines which result in large variances and/or high failure rates of assays using said cell lines.

Assays for determining the biological activity of Clostridial Neurotoxins described in the art include Western blot analysis in which the Neurotoxin activity is quantified by the amount of cleaved Neurotoxin substrate in cell lysates. In other assays, the activity of Clostridial Neurotoxins is measured by an electrochemiluminescence (ECL) sandwich ELISA; see WO 2009/114748 A1. Also in this case, the biological activity of the Clostridial Neurotoxin is determined by the detection of cleaved Clostridial Neurotoxin substrate after isolation from the cell lysate. Further, the Neurotoxin substrate has to be concentrated, in both assays.

In light of the above, further test systems for the determination of Neurotoxin polypeptide activity acceptable to governmental agencies and/or providing for an alternative to animal-based test systems are highly desirable.

Thus, the technical problem underlying the present invention may be seen as the provision of means and methods complying with the aforementioned needs. The technical problem is solved by the embodiments characterized in the claims and herein below.

The present invention relates, in a first aspect, to a method for directly determining the biological activity of a Neurotoxin polypeptide in cells, comprising the steps of:
a) incubating cells susceptible to Neurotoxin intoxication with a Neurotoxin polypeptide for a time and under conditions which allow for the Neurotoxin polypeptide to exert its biological activity;
b) fixing the cells and, optionally, permeabilizing the cells with a detergent;
c) contacting the cells with at least a first capture antibody specifically binding to the non-cleaved and Neurotoxin-cleaved substrate and with at least a second capture antibody specifically binding to the cleavage site of the Neurotoxin-cleaved substrate, under conditions which allow for binding of said capture antibodies to said substrates;
d) contacting the cells with at least a first detection antibody specifically binding to the first capture antibody, under conditions which allow for binding of said first detection antibody to said first capture antibody, thus forming first detection complexes and at least a second detection antibody specifically binding to the second capture antibody, under conditions which allow for binding of said second detection antibody to said second capture antibody, thus forming second detection complexes;
e) determining the amount of the first and second detection complexes of steps d); and
f) calculating the amount of substrate cleaved by said Neurotoxin polypeptide in said cells by means of the second detection complexes, thereby determining the biological activity of said Neurotoxin polypeptide in said cells.

The method of the invention allows for the direct determination of the biological activity of a Neurotoxin polypeptide in cells. This means that no lysis of the cells and no isolation or concentration of the cleaved Neurotoxin substrate from cell lysates is necessary any longer, as in the methods described in the art. For example, in the Western blot analysis-based assay of the art, the Neurotoxin substrate is concentrated by the separation and concentration of the components of the respective sample in the SDS polyacrylamide gel. In the aforementioned ECL sandwich ELISA described in the art, the concentration of the Neurotoxin substrate is carried out by using antibodies which bind specifically to the cleaved Neurotoxin substrate on a microtiter plate to which the cell lysate is added. The cleaved Neurotoxin substrate is isolated from the lysate by binding of the mentioned antibody which results in a concentration of said cleaved Clostridial Neurotoxin substrate. In contrast, the cleaved Neurotoxin substrate, as exemplified for SNAP-25, can be directly detected in the cell, in the method of the invention. To this end, cells which are susceptible to Neurotoxin intoxication as defined in more detail elsewhere herein are incubated with a Neurotoxin polypeptide for a time and under conditions which allow for the Neurotoxin polypeptide to exert its biological activity. In a next step, the cells are fixed, for example, by addition of a fixation agent such as methanol, ethanol, acetone, formaldehyde or mixtures of the mentioned fixation agents. Optionally, the cells can be permeabilized by using at least one detergent as defined elsewhere herein such as Triton X-100, Tween 20, Saponin, Digitonin or n-Octyl-β-glucopyranoside. The detergent can be comprised in an appropriate buffer such as PBS. Thereafter, the cells are contacted with at least a first capture antibody which specifically binds to the non-cleaved and Neurotoxin-cleaved substrate and with at least a second capture antibody specifically binding to the cleavage site of the Neurotoxin-cleaved substrate, under conditions which allow for binding of said capture antibodies to said substrates. Herein, the first capture antibody is able to determine the total content or amount of Neurotoxin substrate in the cells, by binding specifically to an appropriate epitope present in both the non-cleaved and Neurotoxin-cleaved Neurotoxin substrate. The second capture antibody recognizes and binds specifically to an epitope present only in the cleaved Neurotoxin substrate, for example, by binding specifically to the Neurotoxin-cleaved site in the Neurotoxin substrate. Alternatively, the cells can be contacted with a mixture of said first and second capture antibodies, i.e. the cells are contacted with at least a first capture antibody and at least a second capture antibody simultaneously, under the mentioned conditions. In the next step, the cells are contacted with at least a first detection antibody specifically binding to the first capture antibody under conditions which allow for binding of said first detection antibody to said first capture antibody, thus forming first detection complexes. In a subsequent step, the cells are contacted with at least a second detection antibody specifically binding to the second capture antibody, under conditions which allow for binding of said second detection antibody to said second capture antibody, thus forming second detection complexes.

Alternatively, the cells can be contacted with a mixture of said first and second detection antibodies, i.e. the cells are contacted with at least a first detection antibody and at least a second detection antibody simultaneously, under the mentioned conditions. Alternatively, after permeabilization of the cells, they can be contacted with a mixture of said first and second capture antibodies and said first and second detection antibodies simultaneously, under the mentioned conditions. In the next step, the amounts of the first and second detection complexes are determined. Finally, the amount of substrate cleaved by said Neurotoxin polypeptide in said cells is calculated by means of the second detection complexes. Thereby, the biological activity of said Neurotoxin polypeptide is determined directly in the cells.

In the following, the method of the invention is described in more detail. For cell culture, the cells susceptible to Neurotoxin intoxication as defined herein, such as neuronal cells, SiMa cells or iPS-derived neurons, are first seeded on 96 well microtiter plates. SiMa cells are differentiated to a neuronal phenotype, for example, according to the procedures disclosed in WO 2010/105234, and iPS-derived neurons are differentiated to a neuronal phenotype, e.g., according to assays described in WO 2012/135621. Then, the cells are intoxicated with a Neurotoxin polypeptide, such as BoNT/A, for about 72 hours. In the subsequent step, the cells are fixed on the microtiter plate, prior to the ELISA assay. For fixing the cells, for example ice-cold methanol (−20° C.) can be added to the cells for 20 minutes at −20° C.

For performing the ELISA assay, the cells are first washed. As a wash buffer, e.g., 0.1% Triton X-100 in 10 mM PBS buffer (pH 7.4) can be used. Thereafter, endogenous proteases are quenched by a quenching buffer such as 0.6% H₂O₂ in 10 mM PBS (pH 7.4), followed by another wash step. In the following step, free binding sites on the microtiter plate are blocked by an appropriate blocking buffer such as, for instance, 2% BSA in 10 mM PBS buffer (pH 7.4) and 0.05% Triton X-100. Then, the cells are permeabilized, by using an appropriate detergent. As a permeabilization buffer, e.g., 0.5% Triton X-100 in 10 mM PBS buffer can be utilized. Permeabilization allows the diffusion of the antibodies through the pores formed in the cells. Thereafter, the cells are washed by washing buffer as mentioned above.

In the next step, the permeabilized cells are incubated, e.g., with a mixture of two different antibodies. The mixture comprises a first capture antibody specifically binding to the non-cleaved and Neurotoxin-cleaved substrate and a second capture antibody specifically binding to the cleavage site of the Neurotoxin-cleaved substrate. Said first and second capture antibodies can also be applied subsequently. For example, the first capture antibody can specifically bind to both non-cleaved and Neurotoxin-cleaved SNAP-25, thereby allowing for the quantification of the total amount or content of SNAP-25 in the cells. Further, this first capture antibody can be used for the normalization of the amount of cleaved SNAP-25 in the cells, upon evaluation as described herein. The second capture antibody specifically binds to the cleavage site of the Neurotoxin-cleaved substrate and therefore allows the determination and detection of the cleaved Neurotoxin substrate, such as BoNT/A-cleaved SNAP-25.

The following detection of the total Neurotoxin substrate and the Neurotoxin-cleaved Neurotoxin substrate in the method of the invention can be carried out directly on the microtiter plate or cell culture dish, i.e. within the cells. Advantageously, it is therefore not necessary to prepare cell extracts and to isolate and/or concentrate the Neurotoxin substrate from the cell lysate in the method of the invention, as in the methods described in the art. Thereafter, the cells are washed in order to remove excess antibody not bound to the respective antigen. In the subsequent step, the permeabilized cells are contacted with at least a first detection antibody and at least a second detection antibody. Said antibodies can be applied as a mixture, i.e. simultaneously, or subsequently. The first detection antibody specifically binds to the first capture antibody. Thereby, first detection complexes are being formed. The first detection antibody can be directed against the species from which the first capture antibody is derived from. For example, in case the rabbit polyclonal anti-SNAP-25 antibody S9684 (Sigma) is used as a first capture antibody specifically binding to the non-cleaved and BoNT/A-cleaved substrate SNAP-25, an anti-rabbit alkaline phosphatase-conjugated antibody can be used as a first detection antibody. The second detection antibody specifically binds to the second capture antibody. Thereby, second detection complexes are being formed. The second detection antibody can be directed against the species from which the second capture antibody is derived from. For instance, in case the mouse monoclonal antibody (mAb) 20-2-5 of the invention described elsewhere herein is used as a second capture antibody specifically binding to the BoNT/A-cleaved SNAP-25, an anti-mouse horseradish peroxidase (HRP)-conjugated antibody can be used as a second detection antibody. It is evident to those skilled in the art that the first detection antibody and the second detection antibody are conjugated with different enzymes in order to allow for the specific detection of the respective first and second capture antibody as used in the method of the invention. For instance, the HRP-based detection as described elsewhere herein can be used for the BoNT/A-cleaved SNAP-25 and the alkaline phosphatase-based detection for the total (BoNT/A-cleaved and non-cleaved) SNAP-25. Thereafter, the cells are washed again. In a subsequent step, a fluorogenic HRP substrate is added to the cells. As a HRP substrate, e.g., Amplex UltraRed (Invitrogen) can be used which is excited at 540 nm and which emits at 600 nm. Incubation with the HRP substrate is carried out for a time sufficient for sufficient conversion of substrate by the horseradish peroxidase. Subsequent to the incubation with the HRP substrate, for example, the AP substrate DiFMUP (6,8-difluoro-4-methylumbelliferyl phosphate; excitation 360 nm; emission 450 nm) can be added to the HRP substrate and the cells are incubated with a mixture of said two substrates. Incubation with said AP substrate is carried out for a time which allows for sufficient conversion of substrate by the alkaline phosphatase. As known in the art, a substrate has to be converted in an amount which is sufficient so that the measured signal is at least as high as the mean value of the blank plus three standard deviations of the mean, according to the definition of limit of detection. The limit of detection can be determined as described in the literature; see, e.g., Armbruster and Pry, Clinical Biochem. Rev. 2008, 29 (Supplement 1): S49-S52. Because the pH optimum of the alkaline phosphatase is in the alkaline region, the corresponding substrate buffer is strongly alkaline. If the alkaline phosphatase substrate is added to the HRP substrate, the reaction of the horseradish peroxidase is stopped by the alkaline pH and the alkaline phosphatase converts DiFMUP. Converted HRP substrate is not influenced by the alkaline pH. Finally, the fluorescence of the two substrates is measured as follows:

Amplex UltraRed: Excitation 540 nm; emission 600 nm
DiFMUP: Excitation 360 nm; emission 450 nm
As appreciated by those skilled in the art, only those fluorogenic substrates are appropriate for detection of the first and second capture antibody in the method of the invention which exhibit different excitation/emission wave lengths of the used substrates. Only in this case, they allow for the specific detection of each antigen, i.e. the total Neurotoxin substrate (such as non-cleaved and Neurotoxin-cleaved SNAP-25) and the cleaved Neurotoxin substrate (such as Neurotoxin-cleaved SNAP-25). Thereby, it is possible to quantify the total content of Neurotoxin substrate and the content of cleaved Neurotoxin substrate in every well or cell culture dish at the same time. In light of this, it is advantageously possible to automatize the method of the invention. As set forth elsewhere herein it is envisaged that the fluorogenic substrates chosen for the method of the invention exhibit a sufficient shift between the excitation/emission spectra in order to allow for the specific detection of the respective substrate. This requirement is fulfilled, for example, for the HRP substrate Amplex and its derivatives and for the AP substrate DiFMUP. Whereas, in an optimal case, there is no overlap between the excitation/emission spectra of the used fluorogenic substrates, it has been experienced that an overlap of up to 30% in the peak area of the excitation spectra of the used fluorogenic substrates is tolerable.

As further acknowledged by those skilled in the art, the method of the present invention allows for the direct detection and quantification of Neurotoxin substrate cleaved by the Neurotoxin polypeptide in the cells, thereby determining the biological activity of said Neurotoxin polypeptide in said cells. Advantageously, the method of the invention does not require the preparation of cell lysates or extracts and the isolation or concentration of the cleaved Neurotoxin substrate from the cell lysates/extracts, which is necessary for the methods known in the art. As a consequence of this, sample material can be saved. Further, the sample preparation and the number of samples can be reduced by the method of the invention since the amount of total Neurotoxin substrate and the amount of cleaved Neurotoxin substrate in the sample can be determined at the same time. In the assays described in the art, the samples have to be subdivided in order to detect both antigens, i.e. total Neurotoxin substrate and cleaved Neurotoxin substrate, separately from each other. The method of the invention renders the subdivision of the sample unnecessary. Thereby, inhomogeneities resulting from the subdivision of samples can be avoided and sample material can be saved. Furthermore, antigens can be degraded in the assays described in the art which can falsify the detection of the cleaved Neurotoxin substrate. This is because in the assays described in the art, the cells are incubated with detergent-containing lysis buffers which, however, are not able to inactivate the Neurotoxin polypeptide or other endogenous proteases resulting in degradation of the Neurotoxin substrate upon longer storage of the samples. Stronger lysis buffers cannot be used in the ECL sandwich ELISA described in the prior art due to the required use of the cell lysate in said assay. This is because the aggregation of the above-mentioned antigens can result in unspecific adsorption of the antigens to the plastic surface of the cell culture dishes or microtiter plates which in turn disturbs the detection of the antigens by appropriate antibodies. Since the antibodies for the detection of the antigens get into contact with the lysate, too, the antibodies can also aggregate. In this case, no reliable and accurate detection of the antigen is possible anymore. The present inventors have experienced such degradation reactions by using Western blot assays for the detection of the biological activity of Neurotoxin activity described in the art. Upon longer storage of lysates at −20° C., in comparison to fresh lysate samples the detection signal of total SNAP-25 has been found to be strongly reduced and the ratio of cleaved Neurotoxin substrate SNAP-25 to un-cleaved Neurotoxin substrate SNAP-25 had shifted due to degradation processes during the freezing. It has been found by the present inventors that the degradation of the Neurotoxin substrate and/or the instability of the samples can be avoided by directly fixing the cells on the cell culture dish because both the Neurotoxin substrate and the Neurotoxin or other endogenous proteases are inactivated immediately by aggregation on the cell culture dish. This can be achieved by using, for example, fixing of the cells by methanol or other fixatives or fixation agents known in the art, such as ethanol, acetone, formaldehyde or mixtures thereof or other fixation agents described herein. The analysis of the stability of, e.g., parental SiMa cells (human neuroblastoma cells; DSMZ no.: ACC 164) and iPS-derived neurons (Whitemarsh et al. (2012), Toxicol. Sci. 126, 426-35) using this fixation method did not reveal any differences between fresh and cell culture dishes stored seven days in the refrigerator.

As used herein, the singular forms “a”, “an” and “the” include both singular and plural reference unless the context clearly dictates otherwise. By way of example, “a cell” refers to one or more than one cell.

As used herein, the term “about” when qualifying a value of a stated item, number, percentage, or term refers to a range of plus or minus 10 percent, 9 percent, 8 percent, 7 percent, 6 percent, 5 percent, 4 percent, 3 percent, 2 percent or 1 percent of the value of the stated item, number, percentage, or term. Preferred is a range of plus or minus 10 percent.

The terms “comprising”, “comprises” and “comprised of” as used herein are synonyms with “including”, “includes” or “containing”, “contains”, and are inclusive or open-ended and do not exclude additional, non-recited members, elements or method steps. Evidently, the term “comprising” encompasses the term “consisting of”. More specifically, the term “comprise” as used herein means that the claim encompasses all the listed elements or method steps, but may also include additional, unnamed elements or method steps. For example, a method comprising steps a), b) and c) encompasses, in its narrowest sense, a method which consists of steps a), b) and c). The phrase “consisting of” means that the composition (or device, or method) has the recited elements (or steps) and no more. In contrast, the term “comprises” can encompass also a method including further steps, e.g., steps d) and e), in addition to steps a), b) and c).

In case numerical ranges are used herein such as “in a concentration between 1 and 5 micromolar”, the range includes not only 1 and 5 micromolar, but also any numerical value in between 1 and 5 micromolar, for example, 2, 3 and 4 micromolar.

The term “in vitro” as used herein denotes outside, or external to, the animal or human body. The term “in vitro” as used herein should be understood to include “ex vivo”. The term “ex vivo” typically refers to tissues or cells removed from an animal or human body and maintained or propagated outside the body, e.g., in a culture vessel. The term “in vivo” as used herein denotes inside, or internal to, the animal or human body.

The term “Neurotoxin polypeptide” as used herein denotes Clostridium botulinum and Clostridium tetani Neurotoxins, i.e. Botulinum toxins (BoNTs) and Tetanus toxin (TeNT). More specifically, said term encompasses BoNT/A, BoNT/B, BoNT/C1, BONT/D, BONT/E, BoNT/F, BoNT/G, and Tetanus Neurotoxin (TeNT). The Neurotoxin polypeptide and, in particular, its light chain and heavy chain are derivable from one of the antigenically different serotypes of Botulinum Neurotoxins indicated above. In an aspect, said light and heavy chain of the neurotoxin polypeptide are the light and heavy chain of a neurotoxin selected from the group consisting of: BoNT/A, BoNT/B, BoNT/C1, BONT/D, BONT/E, BONT/F. BoNT/G or TeNT. In another aspect, the polynucleotide encoding said Neurotoxin polypeptides comprises a nucleic acid sequence as shown in SEQ ID NO: 1 (BoNT/A), SEQ ID NO: 3 (BoNT/B), SEQ ID NO: 5 (BoNT/C1), SEQ ID NO: 7 (BoNT/D), SEQ ID NO: 9 (BoNT/E), SEQ ID NO: 11 (BoNT/F), SEQ ID NO: 13 (BoNT/G) or SEQ ID NO: 15 (TeNT). Moreover, encompassed is, in an aspect, a polynucleotide comprising a nucleic acid sequence encoding an amino acid sequence as shown in any one of SEQ ID NO: 2 (BoNT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), SEQ ID NO: 14 (BoNT/G) or SEQ ID NO: 16 (TeNT). Further encompassed is in an aspect of the means and methods of the present invention, a Neurotoxin polypeptide comprising or consisting of an amino acid sequence selected from the group consisting of: SEQ ID NO: 2 (BoNT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), SEQ ID NO: 14 (BoNT/G) and SEQ ID NO: 16 (TeNT).

In another aspect, the said polynucleotide is a variant of the aforementioned polynucleotides comprising one or more nucleotide substitutions, deletions and/or additions which in still another aspect may result in a polypeptide having one or more amino acid substitutions, deletions and/or additions. Moreover, a variant polynucleotide of the invention shall in another aspect comprise a nucleic acid sequence variant being at least 40%, at least 50%, at least 60%, at least 70%, at least 75%, at least 80%, at least 85%, at least 90%, at least 95%, at least 96%, at least 97%, at least 98% or at least 99% identical to the nucleic acid sequence as shown in any one of SEQ ID NOs: 1, 3, 5, 7, 9, 11, 13 or 15 or a nucleic acid sequence variant which encodes an amino acid sequence being at least 40%, at least 50%, at least 60%, at least 70%, at least 75%, at least 80%, at least 85%, at least 90%, at least 95%, at least 96%, at least 97%, at least 98% or at least 99% identical to the amino acid sequence as shown in any one of SEQ ID NOs: 2, 4, 6, 8, 10, 12, 14, or 16. The term “identical” as used herein refers to sequence identity characterized by determining the number of identical amino acids between two nucleic acid sequences or two amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wisconsin, USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an aspect, each of the aforementioned variant polynucleotides encodes a polypeptide retaining one or more and, in another aspect, all of the biological properties of the respective Neurotoxin polypeptide, i.e. the BoNT/A, BONT/B, BONT/C1, BONT/D, BONT/E, BONT/F, BoNT/G or Tetanus Neurotoxin (TeNT). Those of skill in the art will appreciate that full biological activity is maintained only after proteolytic activation, even though it is conceivable that the unprocessed precursor can exert some biological functions or be partially active. “Biological properties” as used herein refers to (a) receptor binding, (b) internalization, (c) translocation across the endosomal membrane into the cytosol, and/or (d) endoproteolytic cleavage of proteins involved in synaptic vesicle membrane fusion. In vivo assays for assessing biological activity include the mouse LD50 assay and the ex vivo mouse hemidiaphragm assay as described by Pearce et al. (Pearce 1994, Toxicol. Appl. Pharmacol. 128: 69-77) and Dressler et al. (Dressler 2005, Mov. Disord. 20:1617-1619, Keller 2006, Neuroscience 139: 629-637). The biological activity is commonly expressed in Mouse Units (MU). As used herein, 1 MU is the amount of neurotoxic component, which kills 50% of a specified mouse population after intraperitoneal injection, i.e. the mouse i.p. LD50. In a further aspect, the variant polynucleotides can encode Neurotoxins having improved or altered biological properties, e.g., they may comprise cleavage sites which are improved for enzyme recognition or may be improved for receptor binding or any other property specified above.

Accordingly, the term “biological activity of a Neurotoxin polypeptide” as used herein means the biological properties characteristic for a Neurotoxin polypeptide, namely, a) receptor binding, (b) internalization, (c) translocation across the endosomal membrane into the cytosol, and/or (d) endoproteolytic cleavage of proteins involved in synaptic vesicle membrane fusion. It is envisaged that the Neurotoxin polypeptide as used herein exhibits at least one of the properties a) to d) mentioned above, preferably endoproteolytic cleavage of proteins involved in synaptic vesicle membrane fusion, or two or three or all four biological properties listed in a) to d).

Aspects of the present disclosure comprise, in part, a cell from an established cell line. As used herein, the term “cell” refers to any eukaryotic cell susceptible to Neurotoxin intoxication by a Neurotoxin such as, e.g., BoNT/A, or any eukaryotic cell that can uptake a Neurotoxin. The term cell encompasses cells from a variety of organisms, such as, e.g., murine, rat, porcine, bovine, equine, primate and human cells; from a variety of cell types such as, e.g., neuronal and non-neuronal; and can be isolated from or part of a heterogeneous cell population, tissue or organism. As used herein, the term “established cell line” is synonymous with “immortal cell line,” or “transformed cell line” and refers to a cell culture of cells selected for indefinite propagation from a cell population derived from an organism, tissue, or organ source. By definition, an established cell line excludes a cell culture of primary cells. As used herein, the term “primary cells” are cells harvested directly from fresh tissues or organs and do not have the potential to propagate indefinitely. For example, primary neuronal cells can be used in the method of the invention. An established cell line can comprise a heterogeneous population of cells or a uniform population of cells. An established cell line derived from a single cell is referred to as a clonal cell line. An established cell line can be one whose cells endogenously express all component necessary for the cells to undergo the overall cellular mechanism whereby a Neurotoxin, such as BoNT/A, proteolytically cleaves a substrate, such as SNAP-25, and encompasses the binding of a Neurotoxin to a Neurotoxin receptor, such as BoNT/A, to a BoNT/A receptor, the internalization of the neurotoxin/receptor complex, the translocation of the Neurotoxin light chain from an intracellular vesicle into the cytoplasm and the proteolytic cleavage of a Neurotoxin substrate. Alternatively, an established cell line can be one whose cells have had introduced from an exogenous source at least one component necessary for the cells to undergo the overall cellular mechanism whereby a Neurotoxin, such as BoNT/A, proteolytically cleaves a substrate, such as SNAP-25, and encompasses the binding of a Neurotoxin to a receptor, such as BoNT/A to a BoNT/A receptor, the internalization of the neurotoxin/receptor complex, the translocation of the Neurotoxin light chain from an intracellular vesicle into the cytoplasm and the proteolytic cleavage of a Neurotoxin substrate. Also referred to as a genetically-engineered cell line, cells from such an established cell line may, e.g., express an exogenous FGFR2, an exogenous FGFR3, an exogenous SV2, an exogenous Neurotoxin substrate such as SNAP-25, or any combination thereof.

The term “cell(s) susceptible to Neurotoxin intoxication” as denoted herein means a cell that can undergo the overall cellular mechanisms whereby a Neurotoxin polypeptide (e.g., BoNT/A) cleaves a Neurotoxin substrate (e.g., the BoNT/A substrate SNAP-25) and encompasses the binding of the Neurotoxin to its corresponding receptor (e.g., binding of BoNT/A to the BoNT/A receptor), the internalization of the Neurotoxin/receptor complex, the translocation of the Neurotoxin light chain from an intracellular vesicle into the cytoplasm and the proteolytic cleavage of the Neurotoxin substrate. Assays for determining the biological activity of Neurotoxin polypeptides are well known in the art and also described elsewhere herein; see, e.g., Pellett et al. (2011), Biochem. Biophys. Res. Commun. 404, 388-392; Whitemarsh et al. (2012), Toxicol. Sci. 126, 426-35. Accordingly, a “cell susceptible to Neurotoxin intoxication” as used herein means a Neurotoxin sensitive cell. The mentioned term comprises a cell or a cell line, for example, an isolated, primary cell or a cell line thereof or a cell of an established cell line or an established cell line, for example, tumor cells or tumor cell lines which are capable of differentiating to neuronal cells, such as neuroblastoma cells or neuroblastoma cell lines as defined elsewhere herein. For example, said neuroblastoma cell line can be a SiMa cell line which is commercially available from DSMZ (ACC 164). Specific clones of the cell line SiMa are furthermore disclosed in WO 2010/105234. Other neuroblastoma cell lines which can be used in the method of the invention can be obtained from ATCC or DSMZ, under the following ATCC or DSMZ numbers: Cell line N1E-115 under CRL-2263, cell line Neuro2a under CCL-131, cell line SH-SY5Y under CRL-2266, cell line PC12 under CRL-1721, cell line MHH-NB-11 under ACC 157 (DSMZ) and cell line SK-N-BE(2) under CRL-2271. Other tumor cells which are susceptible to Neurotoxin intoxication are P-19 cells (murine embryonal carcinoma cell line) (DSMZ no. ACC 316). Further encompassed by cells susceptible to Neurotoxin intoxication are induced pluripotent stem cell (iPS)-derived neurons, preferably human induced pluripotent stem cell (iPS)-derived neurons; see, e.g., Whitemarsh et al. (2012), loc. cit. Such human iPS-derived neurons are also commercially available, for instance, from Cellular Dynamics. Methods of generating iPS cells are described, for example, in Yu et al. (Science 2009 May 8; 324(5928): 797-801. Epub 2009), WO 2011/056971 and WO 2011/025852. In some aspects, iPS are differentiated into neurons using suitable methods, e.g., those described in WO 2012/135621 and U.S. Patent Applications US 2010/0279403 and US 2010/0216181.

The term “fixing the cells” means fixing the cells using methods described in the art. Generally, fixation is a chemical process by which biological tissues are preserved from decay, thereby preventing autolysis. Fixation terminates any ongoing biochemical reactions, and may also increase the mechanical strength or stability of the treated tissues. Fixation preserves a sample of biological material such as a tissue or cells as close to its natural state as possible in the process of preparing said tissue or cells for examination or analysis. To this end, a fixative usually acts to disable intrinsic biomolecules—particularly proteolytic enzymes—which otherwise digests or damages the sample. Further, a fixative typically protects a sample from extrinsic damage. Fixatives are toxic to most common microorganisms including bacteria that might exist in a tissue or cell culture or which might otherwise colonize the fixed tissue or cell culture. In addition, many fixatives chemically alter the fixed material to make it less palatable either indigestible or toxic to opportunistic microorganisms. Finally, fixatives often alter the cells or tissues on a molecular level to increase their mechanical strength or stability. This increased strength and rigidity can help preserve the morphology such as shape and structure of the sample as it is processed for further analysis. It is evident to those skilled in the art that the choice of fixative and fixation protocol may depend on the additional processing steps and final analyses that are planned. For example, immunohistochemistry uses antibodies that bind specifically to a specific protein target, the antigen. Prolonged fixation can chemically mask these targets and prevent antibody binding. In these cases, for example, a quick fixation method using cold formalin can be used. Alternatively, the cells can be fixed by adding ice-cold methanol (−20° C.). Besides aldehydes such as formaldehyde or glutaraldehyde and alcohols such as ethanol or methanol, oxidizing agents, Hepes-glutamic acid buffer-mediated organic solvent protection effect (HOPE) fixative, acetone, or mixtures thereof, such as a mixture of methanol and acetone, methanol and ethanol, paraformaldehyde and Triton X-100, or paraformaldehyde and methanol, can be used in fixation protocols. In one aspect of the method of the invention, fixing the cells is carried out by the addition of a fixation agent selected from the group consisting of: methanol, ethanol, acetone, formaldehyde or mixtures thereof. To ensure and/or support free access of the antibody to its antigen, the cells can, optionally, be permeabilized by using an appropriate permeabilization buffer comprising at least one detergent, such as Triton X-100. A permeabilization buffer which can be used in the method of the invention is, e.g., 0.5% Triton X-100 in 10 mM PBS buffer. In other aspects of the methods of the invention, the cells can be permeabilized by using a permeabilization buffer such as PBS comprising at least one detergent selected from Tween 20, Saponin. Digitonin or n-Octyl-β-glucopyranoside. In other aspects, mixtures of two or more of the detergents mentioned herein can be used in the said permeabilization buffer. In general, fixation strengths and times are considerably shorter for cells than on the thicker, structurally complex tissue sections. For immunocytochemistry, sample preparation essentially entails fixing the target cells to the slide, cell culture dish or microtiter plate. Perfect fixation would immobilize the antigens, while retaining authentic cellular and subcellular architecture and permitting unhindered access of antibodies to all cells and subcellular compartments. Wide ranges of fixatives as exemplified above are commonly used, and the correct choice of method will depend on the nature of the antigen being examined and on the properties of the antibody used. Fixation methods fall generally into two classes: organic solvents and cross-linking reagents. Organic solvents such as alcohols and acetone remove lipids and dehydrate the cells, while precipitating the proteins on the cellular architecture. Cross-linking reagents such as paraformaldehyde form intermolecular bridges, normally through free amino groups, thus creating a network of linked antigens. Cross-linkers preserve cell structure better than organic solvents, but may reduce the antigenicity of some cell components, and often require the addition of a permeabilization step as indicated above, to allow access of the antibody to the specimen. Fixation with both methods may denature protein antigens, and for this reason, antibodies prepared against denatured proteins may be more useful for cell staining. The appropriate fixation method should be chosen according to the relevant application. Fixing methods of cells are well described in the art (see, e.g., Methods in cell biology, Volume 37: Antibodies in cell biology; Edited by David J. Asai; 1993, Academic Press Inc.).

The term “contacting” as used in accordance with the method of the invention means bringing the cells and the respective antibodies in physical proximity as to allow physical and/or chemical interaction. Suitable conditions which allow for specific interaction are well known to the person skilled in the art. Evidently, said conditions will depend on the antibodies and the cells to be applied in the method of the present invention and can be adapted routinely by the person skilled in the art. Moreover, a time being sufficient to allow interaction can also be determined by the skilled worker without further ado. It is to be understood that between the individual steps of contacting the cells and the respective antibodies recited in the method of the present invention, washing steps may be performed in order to obtain suitable conditions for contacting. For example, after contacting the cells with at least a first capture antibody specifically to the non-cleaved and Neurotoxin-cleaved substrate and with at least a second capture antibody specifically binding to the cleavage site of the Neurotoxin-cleaved substrate in step c) of the method of the invention, a washing step can be incorporated to remove the remaining solution and/or excess first and second capture antibody, prior to applying the first detection antibody and/or second detection antibody. Similarly, after bringing the cells into contact with the first and/or second detection antibody in the method of the invention, a wash step can be included. An appropriate wash buffer is, for example, 0.1% Triton X-100 in 10 mM PBS buffer (pH 7.4). More specifically, the term “contacting” as used herein, refers to bringing the cells into contact with at least a first capture antibody specifically binding to the non-cleaved and Neurotoxin-cleaved substrate and with at least a second capture antibody specifically binding to the cleavage site of the Neurotoxin-cleaved substrate, under conditions which allow for binding of said capture antibodies to said substrates, in step c) of the method of the invention. The first and second capture antibody can be applied to the cells simultaneously, for example, as a mixture, or subsequently. “Contacting” further refers to bringing into contact the cells with at least a first detection antibody specifically binding to the first capture antibody, under conditions which allow for binding of said first detection antibody to said first capture antibody, and at least a second detection antibody specifically binding to the second capture antibody, under conditions which allow for binding of said second detection antibody to said second capture antibody, in step d) of the method of the invention. Thereby, first and second detection complexes are being formed. Alternatively, the first and second detection antibodies can also be applied subsequently.

As used herein, the term “antibody” refers to a molecule generated by an immune system that was made in response to a particular antigen that specifically binds to that antigen, and includes both naturally occurring antibodies and non-naturally occurring antibodies. An “antibody” as used herein encompasses a monoclonal antibody, a polyclonal antibody, a single chain antibody, a dimer or a multimer, a chimerized antibody, a bispecific antibody, a bispecific single chain antibody, a multispecific antibody, a synthetic antibody, a humanized antibody, a bifunctional antibody, a cell-associated antibody like an Ig receptor, a linear antibody, a diabody, a minibody, or a fragment of any of said antibodies. Fragments of said antibodies include, e.g., Fab, Fv, or scFv fragments, or chemically modified derivatives of any of these fragments. Antibodies can be manufactured by using methods which are described in the art; see, for example, Harlow and Lane “Antibodies, A Laboratory Manual”, CSH Press, Cold Spring Harbor, 1988. Monoclonal antibodies can be prepared by the techniques originally described in Köhler 1975, Nature 256, 495, and Galfré 1981, Meth. Enzymol. 73, 3. Said techniques comprise the fusion of mouse myeloma cells to spleen cells derived from immunized mammals. Antibodies can be further improved by techniques well known in the art. For example, surface plasmon resonance as employed in the Biacore system can be used to increase the efficiency of phage antibodies which bind to the epitope; see, e.g., Schier 1996, Human Antibodies Hybridomas 7, 97; Malmborg 1995. J. Immunol. Methods 183, 7. Antibodies as used herein also comprise functional equivalents of antibodies, i.e. agents which are capable of specifically binding to the desired epitopes or parts of the Neurotoxin substrates. In an aspect, such functional equivalents comprise binding proteins specifically binding to Neurotoxin substrates or domains thereof which are capable of mediating the said specific binding. An antibody as used herein can be a full-length immunoglobulin molecule comprising the VH and VL domains, as well as a light chain constant domain (CL) and heavy chain constant domains, CH1, CH2 and CH3, or an immunologically active fragment of a full-length immunoglobulin molecule, such as, e.g., a Fab fragment, a F(ab′)₂ fragment, a Fc fragment, a Fd fragment, or a Fv fragment. An antibody can be derived from any vertebrate species (e.g., human, goat, horse, donkey, murine, rat, rabbit, or chicken), and can be of any type (e.g., IgG, IgE, IgM, IgD, or IgA), class (e.g., IgA, IgD, IgE. IgG, or IgM) or subclass (IgG1, IgG2, IgG3, IgG4, IgA1 or IgA2). For general disclosure on the structure of naturally occurring antibodies, non-naturally occurring antibodies, and antigenic compound-binding fragments thereof, see, e.g., Plueckthun in The Pharmacology of Monoclonal Antibodies, vol. 113, Rosenburg and Moore eds., Springer-Verlag, New York, pp. 269-315 (1994); Borrabeck, Antibody Engineering 2d ed. (Oxford University Press). Naturally-occurring antibodies are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The complete antigen-recognition and antigen-binding site is contained within the variable domains of the antibody, i.e., the Fv fragment. This fragment includes a dimer of one heavy chain variable domain (VH) and one light chain variable domain (VL) in tight, non-covalent association. Each domain comprises four framework regions (FR), which largely adopting a beta-sheet configuration, connected by three hypervariable regions, which form loops connecting, and in some cases form part of, the beta-sheet structure. Each hypervariable region comprises an amino acid sequence corresponding to a complementarity determining region (CDRs). Collectively, it the three-dimensional configuration of the six CDR regions that define an antigen-binding site on the surface of the VH-VL dimer that confers antigen-binding specificity. See e.g., Cyrus Chothia, et al., Conformations of Immunoglobulin Hypervariable Regions, Nature 342(6252): 877-883 (1989); Elvin A. Kabat, et al Sequences of Proteins of Immunological Interest, 5th Ed. Public Health Service, National Institutes of Health, Bethesda, Md. (1991). The constant domains of the antibody are not involved directly in binding an antibody to an antigen, but exhibit various effector functions, such as participation of the antibody in antibody dependent cellular cytotoxicity.

“Selective binding” or “specific binding” as used herein includes binding properties such as, e.g., binding affinity, binding specificity, and binding avidity; see, e.g., David J. King, Applications and Engineering of Monoclonal Antibodies, pp. 240 (1998). Binding affinity refers to the length of time the antibody resides at its epitope binding site, and can be viewed as the strength with which an antibody binds its epitope. Binding affinity can be described an antibody's equilibrium dissociation constant (KD), which is defined as the ratio Kd/Ka at equilibrium. Ka is the antibody's association rate constant and Kd is the antibody's dissociation rate constant. Binding affinity is determined by both the association and the dissociation and alone neither high association nor low dissociation can ensure high affinity. The association rate constant (Ka), or on-rate constant (Kon), measures the number of binding events per unit time, or the propensity of the antibody and the antigen to associate reversibly into its antibody-antigen complex. The association rate constant is expressed in M⁻¹ s⁻¹, and is symbolized as follows: [Ab]×[Ag]×Kon. The larger the association rate constant, the more rapidly the antibody binds to its antigen, or the higher the binding affinity between antibody and antigen. The dissociation rate constant (Kd), or off-rate constant (Koff), measures the number of dissociation events per unit time propensity of an antibody-antigen complex to separate (dissociate) reversibly into its component molecules, namely the antibody and the antigen. The dissociation rate constant is expressed in s⁻¹, and is symbolized as follows: [Ab+Ag]×Koff. The smaller the dissociation rate constant, the more tightly bound the antibody is to its antigen, or the higher the binding affinity between antibody and antigen. The equilibrium dissociation constant (KD) measures the rate at which new antibody-antigen complexes formed equals the rate at which antibody-antigen complexes dissociate at equilibrium. The equilibrium dissociation constant is expressed in M, and is defined as Koff/Kon=[Ab]×[Ag]/[Ab+Ag], where [Ab] is the molar concentration of the antibody, [Ag] is the molar concentration of the antigen, and [Ab+Ag] is the of molar concentration of the antibody-antigen complex, where all concentrations are of such components when the system is at equilibrium. The smaller the equilibrium dissociation constant, the more tightly bound the antibody is to its antigen, or the higher the binding affinity between antibody and antigen. Thus, in one aspect of the method of the invention, the first capture antibody specifically binding to the non-cleaved and Neurotoxin-cleaved substrate can have an association rate constant of, e.g., less than 1×10⁵ M⁻¹ s⁻¹, less than 1×10⁶ M⁻¹ s⁻¹, less than 1×10⁷ M⁻¹ s⁻¹ or less than 1×10⁸ M⁻¹ s⁻¹. In another aspect, the first capture antibody specifically binding to the non-cleaved and Neurotoxin-cleaved substrate can have an association rate constant of, e.g., more than 1×10⁵ M⁻¹ s⁻¹, more than 1×10⁶ M⁻¹ s⁻¹, more than 1×10⁷ M⁻¹ s⁻¹ or more than 1×10⁸ M⁻¹ s⁻¹. In a further aspect, the first capture antibody specifically binding to the non-cleaved and Neurotoxin-cleaved substrate can have a disassociation rate constant of, e.g., less than 1×10⁻³ M⁻¹ s⁻¹, less than 1×10⁴ M⁻¹ s⁻¹, less than 1×10⁵ M⁻¹ s⁻¹ or less than 1×10⁻⁶ M⁻¹ s⁻¹. In a still further aspect, the first capture antibody specifically binding to the non-cleaved and Neurotoxin-cleaved substrate can have a disassociation rate constant of, e.g., more than 1×10⁻³M⁻¹ s⁻¹, more than 1×10⁴ M⁻¹ s⁻¹, more than 1×10⁻⁵ M⁻¹ s⁻¹ or more than 1×10⁻⁶ M⁻¹ s⁻¹. In a further aspect, the second capture antibody specifically binding to the specifically binding to the cleavage site of the Neurotoxin-cleaved substrate can have an association rate constant of, e.g., less than 1×10⁵ M⁻¹ s⁻¹, less than 1×10⁶ M⁻¹ s⁻¹, less than 1×10⁷ M⁻¹ s⁻¹ or less than 1×10⁸ M⁻¹ s⁻¹. In another aspect, the second capture antibody specifically binding to the specifically binding to the cleavage site of the Neurotoxin-cleaved substrate can have an association rate constant of, e.g., more than 1×10⁵ M⁻¹ s⁻¹, more than 1×10⁶ M⁻¹ s⁻¹, more than 1×10⁷ M⁻¹ s⁻¹ or more than 1×10⁸ M⁻¹ s⁻¹. In a further aspect, the second capture antibody specifically binding to the specifically binding to the cleavage site of the Neurotoxin-cleaved substrate can have a disassociation rate constant of, e.g., less than 1×10⁻³ M⁻¹ s⁻¹, less than 1×10⁴ M⁻¹ s⁻¹, less than 1×10⁻⁵ M⁻¹ s⁻¹ or less than 1×10⁻⁶ M⁻¹ s⁻¹. In a still further aspect, the second capture antibody specifically binding to the specifically binding to the cleavage site of the Neurotoxin-cleaved substrate can have a disassociation rate constant of, e.g., more than 1×10⁻³ M⁻¹ s⁻¹, more than 1×10⁴ M⁻¹ s⁻¹, more than 1×10⁵ M⁻¹ s⁻¹ or more than 1×10⁻⁶ M⁻¹ s⁻¹.

A target antigen such as the Neurotoxin-cleaved or non-cleaved Neurotoxin substrates SNAP-25, VAMP/Synaptobrevin, or Syntaxin generally has one or more binding sites, also called epitopes, which are recognized by the CDR-formed antigen-binding site of the antibody. As used herein, an “epitope” is synonymous with “antigenic determinant” and refers to the site on a target antigen, such as, e.g., a peptide, polypeptide, polysaccharide or lipid-containing molecule, capable of specific binding to an immunoglobulin or T-cell receptor or otherwise interacting with a molecule. Each antibody that specifically binds to a different epitope has a different structure. Thus, one antigen may have more than one corresponding antibody. “Specific binding” as referred to herein can be tested by various well known techniques including, e.g., competition experiments and Western blots. An epitope as used in accordance with the invention relates to the antigenic determinant in the Neurotoxin substrates, e.g. SNAP-25, VAMP/Synaptobrevin, or Syntaxin which is recognized by the antibody. As used herein, the term “specifically” means selectively and refers to having a unique effect or influence or reacting in only one way or with only one thing. As used herein, the term “specifically binds” or “selectively binds” when made in reference to an antibody or binding protein or binding domain, refers to the discriminatory binding of the antibody or binding protein/domain to the indicated target epitope such that the antibody or binding protein/domain does not substantially cross react with non-target epitopes. The minimal size of a peptide epitope, as defined herein, is about five amino acid residues, and a peptide epitope typically comprises at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or at least 30 amino acid residues. A peptide epitope may be a linear or a discontinuous epitope. A discontinuous epitope comprises amino acid residues that are not adjacent in the primary structure of the peptide but are brought together into an epitope by way of the secondary, tertiary or quaternary structure of the peptide. Furthermore, it is also noted that an epitope may comprise a portion of a molecule other than an amino acid sequence such as, e.g., carbohydrate moiety, lipid moiety like glycolipids or lipoproteins, or a chemically modified amino acid moiety like a phosphorylated amino acid.

According to the method of the present invention, the “first capture antibody” specifically binds to an epitope comprised by the non-cleaved and Neurotoxin-cleaved substrate. Said Neurotoxin substrates can be, example, for SNAP-25, VAMP/Synaptobrevin, or Syntaxin. For instance, SNAP-25 is a known substrate of BONT/A, BONT/C1 and BoNT/E. VAMP/Synaptobrevin is a substrate of BoNT/B, BONT/D. BONT/F, BoNT/G and TeNT, whereas Syntaxin is a substrate of BoNT/C1. Said first capture antibody allows for the determination of the total amount, i.e. complete content of the respective Neurotoxin substrate in the cells. For example, in SNAP-25, having a total length of 205 amino acid residues, the cleavage site for BoNT/A is localized between amino acid residues Gln 197 and Arg 198. Accordingly, an antibody specifically binding to an epitope positioned N-terminally to the BoNT/A cleavage site, i.e. an epitope localized between amino acid residues 1 and 198 of SNAP-25 can be used as first capture antibody. For example, said antibody can specifically bind to an N-terminal epitope or an epitope positioned in the mid-part of SNAP-25. For BoNT/C1, an epitope positioned N-terminally to the BoNT/C1 cleavage site (Arg 198-Ala 199), i.e. between amino acid residues 1 and 199 of SNAP-25 can be used as first capture antibody. For BoNT/E, an epitope positioned N-terminally to the BoNT/E cleavage site(Arg 180-Ile 181), i.e. between amino acid residues 1 and 181 of SNAP-25 can be used as first capture antibody. If VAMP is used as a Neurotoxin substrate, an epitope positioned N-terminally to the BoNT/B cleavage site (Gln 76-Phe 77), i.e. between amino acid residues 1 and 77 of VAMP can be used as first capture antibody. An epitope positioned N-terminally to the BoNT/D cleavage site (Lys 59-Leu 60), i.e. between amino acid residues 1 and 60 of VAMP2 can be used as first capture antibody. An epitope positioned N-terminally to the BoNT/F cleavage site (Gln 58-Lys 59), i.e. between amino acid residues 1 and 59 of VAMP2 can be used as first capture antibody. An epitope positioned N-terminally to the BoN/G cleavage site (Ala 81-Ala 82), i.e. between amino acid residues 1 and 82 of VAMP2 can be used as first capture antibody. If Syntaxin is used as a substrate, an epitope positioned N-terminally to the BoN/C1 cleavage site (Lys 253-Ala 254), i.e. between amino acid residues 1 and 254 of Syntaxin 1a can be used as first capture antibody.

A neurotoxin cleavage site recognized and cleaved by the BoNT/A protease, in an aspect of the invention, is derived from a protein that is sensitive to cleavage by BoNT/A. In an aspect, such a protein is human SNAP-25A or SNAP-25B or a homolog, paralog or ortholog thereof from rat, mouse, bovine, Danio, Carassius, Xenopus, Torpedo, Strongylocentrotus, Loligo, Lymnaea or Aplysia. Suitable cleavage sites derived from said proteins are disclosed, e.g., in EP 1 926 744 B1.

A neurotoxin cleavage site recognized and cleaved by the BoNT/B protease, in an aspect of the invention, is derived from a protein that is sensitive to cleavage by BoNT/B. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/C1 protease, in an aspect of the invention, is derived from a protein that is sensitive to cleavage by BoNT/C1. In an aspect, such a protein is human and mouse Syntaxin 1A, Syntaxin 1B1, Syntaxin 2-1, Syntaxin 2-2, Syntaxin 2-3, Syntaxin 3A or Syntaxin 1B2, bovine or rat Syntaxin 1A, Syntaxin 1B1 or Syntaxin 1B2, rat Syntaxin 2 or Rat syntaxin 3, mouse Syntaxin 1A, Syntaxin 1B1, Syntaxin 1B2, Syntaxin 2, Syntaxin 3A, Syntaxin 3B or Syntaxin 3C, chicken Syntaxin 1A or Syntaxin 2; Xenopus Syntaxin 1A or Syntaxin 1B, Danio Syntaxin 1A, Syntaxin 1B or Syntaxin 3, Torpedo Syntaxin 1A or Syntaxin 1B, Strongylocentrotus Syntaxin 1A or Syntaxin 1B, Drosophila Syntaxin 1A or Syntaxin 1B, Hirudo Syntaxin 1A or Syntaxin 1B, Loligo Syntaxin 1A or Syntaxin 1B, Lymnaea Syntaxin 1A or Syntaxin 1B or any ortholog, paralog or homolog thereof. Suitable cleavage sites derived from said proteins are disclosed in EP 1 926 744 B1.

A neurotoxin cleavage site recognized and cleaved by the BoNT/D protease, in an aspect of the invention, is derived from a protein that is sensitive to cleavage by BoNT/D. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/E protease, in an aspect of the invention, is derived from a protein that is sensitive to cleavage by BoNT/E. In an aspect, such a protein is, such a protein is human SNAP-25A or B or a homolog, paralog or ortholog thereof from rat, mouse, bovine, Danio, Carassius, Xenopus, Torpedo, Strongylocentrotus, Loligo, Lymnaea or Aplysia. Suitable cleavage sites derived from said proteins are disclosed in EP 1 926 744 B1.

A neurotoxin cleavage site recognized and cleaved by the BoNT/F protease, in an aspect of the invention, is derived from a protein that is sensitive to cleavage by BoNT/F. In an aspect, such a protein is, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/G protease, in an aspect of the invention, is derived from a protein that is sensitive to cleavage by BoNT/G. In an aspect, such a protein is, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the TeNT protease, in an aspect of the invention, is derived from a protein that is sensitive to cleavage by TeNT. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

Examples for appropriate antibodies which can be used as first capture antibodies in the method of the invention include, for example, the rabbit polyclonal anti-SNAP-25 antibody S9684 (Sigma) (Fernández-Salas E, Wang J, Molina Y, Nelson J B, Jacky B P S, et al. (2012) Botulinum Neurotoxin Serotype a Specific Cell-Based Potency Assay to Replace the Mouse Bioassay. PLOSONE7(11): e49516. doi: 10.1371/journal.pone.0049516), the rabbit polyclonal anti-SNAP25 antibody PA5-19708 (Pierce Antibodies), the rabbit polyclonal anti-SNAP25 antibody PA5-19701 (Pierce Antibodies), the VAMP/Synaptobrevin antibody sc-13992 (Santa Cruz Biotechnology) or #104 203 (Synaptic Systems), or the Syntaxin antibody ADI-VAM-SV013 (Enzo Life Sciences).

In one aspect, the first capture antibody that recognizes the non-cleaved and Neurotoxin-cleaved substrate in order to determine the total amount of Neurotoxin substrate in the cell is used for normalization, as shown in the following Examples.

The “second capture antibody” as used herein specifically binds to the cleavage site of the Neurotoxin-cleaved substrate. Accordingly, said second capture antibody recognizes selectively the Neurotoxin substrate cleaved by the Neurotoxin, for example, the BoNT/A SNAP-25-cleaved product. In contrast, said second capture antibody is not able to bind to the non-cleaved Neurotoxin substrate, such as, e.g., non-cleaved SNAP-25. Examples for appropriate antibodies which can be used as second capture antibodies in the method of the invention include, for example, the mouse monoclonal antibodies of the invention as indicated below, the mouse monoclonal antibody MC-6053 (R&D Systems) which recognizes the BoNT/A-cleaved SNAP-25 ((Baldwin and Barbieri 2007, Biochemistry 46, 3200-3210), as well as the mouse monoclonal antibody DMAB4345 (Creative Diagnostics).

The present invention provides in a further aspect, novel monoclonal antibodies specifically binding to the cleavage site of the Neurotoxin-cleaved SNAP-25, i.e. to Neurotoxin-cleaved SNAP-25 only, whereas they do not bind to non-cleaved SNAP-25. Said monoclonal antibodies have been generated and characterized as described in the following Examples and have been found particularly suitable as second capture antibodies for the method of the invention, due to their high affinity and specificity for Neurotoxin-cleaved SNAP-25. Preferably, the monoclonal antibodies of the invention recognize and specifically bind to the epitope SNAP-25₁₉₀₋₁₉₇ “TRIDEANQ” shown in SEQ ID NO: 74 and/or to SNAP-25₁₉₇, i.e. Neurotoxin (e.g., BoNT/A)-cleaved SNAP-25. More preferably, the monoclonal antibodies of the invention recognize and specifically bind to the epitope SNAP-25₁₉₁₋₁₉₇ “RIDEANQ” shown in SEQ ID NO: 75 and/or to SNAP-25₁₉₇, to the epitope SNAP-25₁₉₂₋₁₉₇ “IDEANQ” of the sequence shown in SEQ ID NO: 76 and/or to SNAP-25₁₉₇, or to the epitope SNAP-25₁₉₃₋₁₉₇ “DEANQ” shown in SEQ ID NO: 77 and/or to SNAP-25₁₉₇.

In one aspect, the present invention relates to an antibody or a fragment thereof, which specifically binds to the cleavage site of the BoNT/A-cleaved SNAP-25 and which comprises a heavy chain variable region (VH) comprising an amino acid sequence shown in SEQ ID NO. 18 and/or a light chain variable region (VL) comprising an amino acid sequence shown in SEQ ID NO. 19. Further encompassed by the invention are antibodies or fragments thereof which comprise one, two or three complementarity determining regions (CDRs) of said heavy chain and/or light chain variable region(s). The corresponding CDRH1. CDRH2 and CDRH3 sequences are shown in SEQ ID NOs. 20 to 22, respectively, whereas the corresponding CDRL1, CDRL2 and CDRL3 sequences are shown in SEQ ID NOs. 23 to 25, respectively. The mentioned sequences correspond to mouse monoclonal antibody 20-2-5 as shown in the following Examples. In addition, the present invention pertains to an antibody or a fragment thereof, which specifically binds to the cleavage site of the BoNT/A-cleaved SNAP-25 and which comprises a heavy chain variable region (VH) comprising an amino acid sequence shown in SEQ ID NO. 26 and/or a light chain variable region (VL) comprising an amino acid sequence shown in SEQ ID NO. 27. Further encompassed by the invention are antibodies or fragments thereof which comprise one, two or three complementarity determining regions (CDRs) of said heavy chain and/or light chain variable region(s). The corresponding CDRH1, CDRH2 and CDRH3 sequences are shown in SEQ ID NOs. 28 to 30, respectively, whereas the corresponding CDRL1, CDRL2 and CDRL3 sequences are shown in SEQ ID NOs. 31 to 33, respectively. The mentioned sequences correspond to mouse monoclonal antibody 5-10-5 as shown in the following Examples. Further, the present invention relates to an antibody or a fragment thereof, which specifically binds to the cleavage site of the BONT/A-cleaved SNAP-25 and which comprises a heavy chain variable region (VH) comprising an amino acid sequence shown in SEQ ID NO. 34 and/or a light chain variable region (VL) comprising an amino acid sequence shown in SEQ ID NO. 35. Further encompassed by the invention are antibodies or fragments thereof which comprise one, two or three complementarity determining regions (CDRs) of said heavy chain and/or light chain variable region(s). The corresponding CDRH1, CDRH2 and CDRH3 sequences are shown in SEQ ID NOs. 36 to 38, respectively, whereas the corresponding CDRL1, CDRL2 and CDRL3 sequences are shown in SEQ ID NOs. 39 to 41, respectively. The mentioned sequences correspond to mouse monoclonal antibody 1-10-4 as shown in the following Examples. The present invention pertains also to an antibody or a fragment thereof, which specifically binds to the cleavage site of the BoNT/A-cleaved SNAP-25 and which comprises a heavy chain variable region (VH) comprising an amino acid sequence shown in SEQ ID NO. 42 and/or a light chain variable region (VL) comprising an amino acid sequence shown in SEQ ID NO. 43. Further encompassed by the invention are antibodies or fragments thereof which comprise one, two or three complementarity determining regions (CDRs) of said heavy chain and/or light chain variable region(s). The corresponding CDRH1, CDRH2 and CDRH3 sequences are shown in SEQ ID NOs. 44 to 46, respectively, whereas the corresponding CDRL1, CDRL2 and CDRL3 sequences are shown in SEQ ID NOs. 47 to 49, respectively. The mentioned sequences correspond to mouse monoclonal antibody 16-5-4 as shown in the following Examples. In addition, the present invention relates to an antibody or a fragment thereof, which specifically binds to the cleavage site of the BoNT/A-cleaved SNAP-25 and which comprises a heavy chain variable region (VH) comprising an amino acid sequence shown in SEQ ID NO. 50 and/or a light chain variable region (VL) comprising an amino acid sequence shown in SEQ ID NO. 51. Further encompassed by the invention are antibodies or fragments thereof which comprise one, two or three complementarity determining regions (CDRs) of said heavy chain and/or light chain variable region(s). The corresponding CDRH1, CDRH2 and CDRH3 sequences are shown in SEQ ID NOs. 52 to 54, respectively, whereas the corresponding CDRL1, CDRL2 and CDRL3 sequences are shown in SEQ ID NOs. 55 to 57, respectively. The mentioned sequences correspond to mouse monoclonal antibody 6-3-8 as shown in the following Examples. The present invention pertains further to an antibody or a fragment thereof, which specifically binds to the cleavage site of the BONT/A-cleaved SNAP-25 and which comprises a heavy chain variable region (VH) comprising an amino acid sequence shown in SEQ ID NO. 58 and/or a light chain variable region (VL) comprising an amino acid sequence shown in SEQ ID NO. 59. Further encompassed by the invention are antibodies or fragments thereof which comprise one, two or three complementarity determining regions (CDRs) of said heavy chain and/or light chain variable region(s). The corresponding CDRH1, CDRH2 and CDRH3 sequences are shown in SEQ ID NOs. 60 to 62, respectively, whereas the corresponding CDRL1, CDRL2 and CDRL3 sequences are shown in SEQ ID NOs. 63 to 65, respectively. The mentioned sequences correspond to mouse monoclonal antibody 18-3-3 as shown in the following Examples. Moreover, the present invention concerns an antibody or a fragment thereof, which specifically binds to the cleavage site of the BoNT/A-cleaved SNAP-25 and which comprises a heavy chain variable region (VH) comprising an amino acid sequence shown in SEQ ID NO. 66 and/or a light chain variable region (VL) comprising an amino acid sequence shown in SEQ ID NO. 67. Further encompassed by the invention are antibodies or fragments thereof which comprise one, two or three complementarity determining regions (CDRs) of said heavy chain and/or light chain variable region(s). The corresponding CDRH1, CDRH2 and CDRH3 sequences are shown in SEQ ID NOs. 68 to 70, respectively, whereas the corresponding CDRL1, CDRL2 and CDRL3 sequences are shown in SEQ ID NOs. 71 to 73, respectively. The mentioned sequences correspond to mouse monoclonal antibody 14-12-1 as shown in the following Examples.

The term “first detection antibody” as used herein is an antibody specifically binding to the first capture antibody. Said first detection antibody allows for the specific detection of the first capture antibody. By measuring the amount of bound first detection antibody, the amount of first detection complexes can be determined since the amount of bound first detection antibody in the first detection complex correlates with the amount of first capture antibody (and accordingly the amount of total, i.e. cleaved and non-cleaved Neurotoxin substrate) comprised by the first detection complex. For example, an appropriate species-specific antibody can be used as a first detection antibody: If a mouse antibody has been used as a first capture antibody, said first detection antibody can be an anti-mouse antibody specifically binding to the mouse antibody. The first detection antibody can be, for instance, an alkaline phosphatase (AP)-conjugated antibody, a horseradish-peroxidase (HRP)-conjugated antibody or an antibody conjugated to a fluorescence dye. Conjugation of enzymes to antibodies, for example, by using glutaraldehyde is well known in the art.

Enzyme linked immunosorbent assays (ELISA) have been used to quantitate a wide range of compounds and pathogens for almost 40 years. Initially, radioactivity was used to quantitate the assays, but radioimmunoassays (RIA) have been replaced with assays utilizing enzymes to obtain colorimetric results. Recently new substrates have been developed to produce fluorescent and luminescent products. The basic tenet of the new assays remains the same as colorimetric assays. The substrate is converted into a measurable compound by the enzymatic activity of proteins conjugated to an antibody, which confers specificity.

Commonly used enzyme conjugates in ELISA are alkaline phosphatase or horseradish peroxidase. Accordingly, in one aspect, the first detection antibody can be, for instance, conjugated to alkaline phosphatase or horseradish peroxidase. Further examples of enzyme conjugates which can be used as a first detection antibody in the method of the invention include glucose oxidase which uses glucose as substrate, tyrosinase which converts the substrate 1-(4-Methyl-coumarin-7-yl)-3-(4-hydroxyphenyl)urea) (PAP-AMC) (Stratis Avrameas, Immunochemistry, Volume 6, Issue 1, January 1969, Pages 43-48, IN9-IN11, 49-52) or β-galactosidase which converts the substrate 6,8-difluoro-4-methylumbelliferyl β-d-galactopyranoside(DiFMUG) (Gee et al., Analytical Biochemistry, Volume 273, Issue 1. August 1999, pages 41-48). Upon addition of a substrate, said substrate is converted by the enzyme to a detectable form. For example, alkaline phosphatase catalyzes the cleavage of esters of phosphoric acid. If an alkaline phosphatase (AP)-conjugated antibody is used as a first detection antibody, an appropriate substrate such as a 4-methylumbelliferryl phosphate derivative, e.g., 6,8-Difluoro-4-methylumbelliferyl phosphate (DiFMUP), or fluorescein diphosphate (FDP). 6,8-difluoro-4-methylumbelliferyl phosphate (DiFMUP) is converted by the AP to a detectable form, i.e. the fluorogenic product 6,8-difluoro-4-methylumbelliferone. Said substrate is provided, e.g., by Molecular Probes. Fluorescence intensities of this reaction product of DiFMUP can be measured using excitation/emission maxima of about 358/450 nm. Further substrates which can be used for this purpose are 9H-(1,3-dichloro-9,9-dimethylacridin-2-one-7-yl) phosphate (DDAO-phosphate; Invitrogen), fluorescein diphosphate (FDP; Sigma Aldrich) or 4-methylumbelliferyl phosphate (MUP; Invitrogen). DDAO-phosphate is converted by the AP to the fluorogenic product dimethylacridinone (DDAO), having an excitation/emission maxima of about 646/659 nm. If FDP is used as substrate for the AP, the reaction product is fluorescein, having an excitation/emission maximum of about 490/514 nm. For MUP, the corresponding reaction product is 4-methylumbelliferone (7-hydroxy-4-methylcoumarin), having an excitation/emission maxima of about 360/449 nm. Also these substrates are commercially available, e.g. from Molecular Probes. Alternatively, horseradish peroxidase can be used as enzyme conjugate in the first detection antibody of the method of the invention. Horseradish peroxidase (HRP) catalyzes the reduction of hydrogen peroxide (H₂O₂) to water (H₂O). In the presence of specific substrates, which act as hydrogen donors, the action of HRP converts colorless or non-fluorescent molecules into colored and/or fluorescent moieties respectively. For instance, Amplex® Red (Life Technologies) is a substrate for use with HRP containing assays. Amplex Red, in the presence of peroxidase enzyme, reacts with H₂O₂ in a 1:1 stoichiometry to produce resorufin, a red fluorescent compound which has an absorption and fluorescence emission maxima of 563 nm and 587 nm, respectively. Another example for a HRP substrate is Amplex® UltraRed (Life Technologies). It has been reported that Amplex® UltraRed reagent (excitation/emission of ˜570/585 nm) improves upon the performance of the Amplex® Red reagent, offering brighter fluorescence and enhanced sensitivity on a per-mole basis in horseradish peroxidase or horseradish peroxidase-coupled enzyme assays. Fluorescence of the oxidized Amplex® UltraRed reagent (Amplex® UltroxRed reagent) is also less sensitive to pH, and the substrate and its oxidation product exhibit greater stability that the Amplex® Red reagent in the presence of hydrogen peroxide (H₂O₂) or thiols such as dithiothreitol (DTT). Further appropriate HRP substrates which can be used in the method of the invention include, e.g., 10-Acetyl-3,7-Dihydroxyphenoxazine (ADHP; AnaSpec) or 3-(4-Hydroxyphenyl) propionic acid (HPPA; AnaSpec) (Tuuminen et al. 1991, J. Immunoassay 12, 29-46).

Alternatively, the first detection antibody can carry an appropriate, detectable label which allows for the detection of the first capture antibody. Labeling may be done by direct or indirect methods. Direct labeling involves binding of the label directly (covalently or non-covalently) to the first detection antibody. Indirect labeling involves binding (covalently or non-covalently) of an agent which specifically binds to the first detection antibody and which carries a detectable label. Such an agent may be, e.g., a secondary (higher order) antibody which specifically binds to the first detection antibody. The secondary antibody in such a case will be coupled to a detectable label. It will be understood that further higher order antibodies can be used in addition for detection of the first detection complex. The higher order antibodies are often used to increase the signal. Suitable higher order antibodies may also include the well-known streptavidin-biotin system (Vector Laboratories, Inc.), and the well-known Dako LSAB™2 and LSAB™+ (labeled streptavidin-biotin), or Dako PAP (Peroxidase Anti-Peroxidase). In a further aspect, the said label of the first detection antibody is a fluorescent dye, i.e. the first antibody is conjugated to a fluorescent dye. In this case, the fluorescence can be directly measured by a fluorescence reader. Typical fluorescent labels include fluorescent proteins such as GFP and its derivatives, Cy dyes such as Cy3, or Cy5, Texas Red, Fluorescein, and the Alexa dyes, e.g. Alexa 568.

The “second detection antibody” as used herein is an antibody specifically binding to the second capture antibody. The second detection antibody can be, for instance, conjugated to an enzyme such as alkaline phosphatase, horseradish peroxidase, glucose oxidase or tyrosinase. Accordingly, in one aspect, the second detection antibody is an alkaline phosphatase (AP)-conjugated antibody, a horseradish-peroxidase (HRP)-conjugated antibody, an glucose oxidase-conjugated antibody or a tyrosinase-conjugated antibody. Said second detection antibody allows for the specific detection of the second capture antibody. By measuring the amount of bound second detection antibody, the amount of second detection complexes can be determined since the amount of bound second detection antibody in the second detection complex correlates with the amount of second capture antibody (and accordingly the amount of cleaved Neurotoxin substrate) comprised by the first detection complex. For example, if a rabbit antibody has been used as a second capture antibody, an anti-rabbit antibody can be used as a second detection antibody. The second detection antibody can carry an enzyme as set forth above or a label such as a fluorescent dye (i.e. the second detection antibody is conjugated to a fluorescent dye) as mentioned elsewhere herein with respect to the first detection antibody. In one aspect of the method of the invention, the enzyme conjugated to the first detection antibody differs from the enzyme conjugated to the second detection antibody in order to allow the specific detection of the respective first and second capture antibody in the method of the invention. For instance, if the first detection antibody is an AP-conjugated antibody, the second detection antibody can be a horseradish peroxidase (HRP)-conjugated antibody or vice versa. Further, the excitation/emission spectra of the fluorogenic substrates of the AP and HRP do not substantially overlap but differ from each other, i.e. they show a clear shift so as to allow the distinction of the fluorescence intensities generated by the respective product. For example, DiFMUP exhibits excitation/emission at ˜358/450 nm, whereas Amplex UltraRed exhibits excitation/emission of ˜570/585 nm, thereby allowing for accurate measurements of the fluorescence intensities generated by the conversion of said fluorogenic substrates by the respective enzyme. In a further aspect, the alkaline phosphatase (AP)-conjugated antibody is used as a first detection antibody for the antigen which is present in excess in the cell, i.e. for the measurement of the amount of the total (non-cleaved and cleaved) Neurotoxin substrate, such as total SNAP-25, in the cell. The horseradish peroxidase (HRP)-conjugated antibody is used as a second detection antibody for the antigen which is present in the cell in a lower amount, i.e. for the measurement of the amount of the cleaved Neurotoxin substrate, such as BoNT/A cleaved SNAP-25, in the cell. As known in the art, HRP substrates are more sensitive than AP substrates meaning that lower amounts of analytes can be detected. If an HRP antibody is used as secondary antibody for the detection of cleaved SNAP-25, lower amounts of cleaved SNAP-25 are detectable. In turn, lower amounts of BoNT/A can be determined, thereby increasing the sensitivity of the assay. Because the AP antibody measures the total amount of SNAP-25 in the cell, high sensitivity for the substrate is not required, due to the excess of analyte.

The term “at least” as used herein such as, for example, “at least a first capture antibody” means that in addition to an antibody specifically binding to the non-cleaved and Neurotoxin-cleaved substrate, one or more further antibodies with the mentioned specificity can be used in the method of the invention. Similarly, “at least a second capture antibody” means that in addition to an antibody specifically binding to the cleavage site of the Neurotoxin-cleaved substrate, one or more further antibodies with the mentioned specificity can be used in the method of the invention. Further, one or more first detection antibodies specifically binding to the first detection antibody (or first detection antibodies) can be used in the method of the invention. Similarly, one or more second detection antibodies specifically binding to the second detection antibody (or second detection antibodies) can be used in the method of the invention.

The term “first detection complex” refers to a complex comprising a first capture antibody and a first detection antibody which specifically binds to the non-cleaved and Neurotoxin-cleaved substrate, thereby allowing for the determination of the total content of Neurotoxin substrate in the cell. The amount of first detection complex can be measured by determination of the amount of specifically bound first detection antibody. This can be achieved dependent on the nature of the enzyme or the label of the first detection antibody, e.g. by measuring the intensity of fluorescence.

The term “second detection complex” refers to a complex comprising the second capture antibody and the second detection antibody which specifically binds to the cleavage site of the Neurotoxin-cleaved substrate, thereby allowing for the determination of the content of cleaved Neurotoxin substrate in the cell. The amount of second detection complex can be measured by determination of the amount of specifically bound second detection antibody. This can be achieved dependent on the nature of the enzyme or the label of the second detection antibody, e.g. by measuring the intensity of fluorescence.

It is envisioned that instead of enzyme-linked immunosorbent analysis (ELISA), any detection system can be used to practice aspects of the method of the invention, with the provision that the signal to noise ratio can distinguish to a statistically significant degree the signal from the formed antibody-antigen complexes from the background signal. Non-limiting examples of immuno-based detection systems include immunoblot analysis, like Western blotting and dot-blotting, immunoprecipitation analysis, and sandwich ELISA. The detection of the signal can be achieved using autoradiography with imaging or phosphorimaging (AU), bioluminescence (BL), fluorescence, resonance energy transfer, plane polarization, colormetric, or flow cytometry (FC). Descriptions of immuno-based detection systems are disclosed, for example, in Commonly Used Techniques in Molecular Cloning, pp. A8.1-A8-55 (Sambrook & Russell, eds., Molecular Cloning A Laboratory Manual, Vol. 3, 3.sup.rd ed. 2001); Detection Systems, pp. A9.1-A9-49 (Sambrook & Russell, eds., Molecular Cloning A Laboratory Manual, Vol. 3, 3.sup.rd ed. 2001).

In a further aspect, the cells, antibodies, Neurotoxin polypeptides and Neurotoxin substrates or any other product as referred to herein are isolated cells, antibodies, Neurotoxin polypeptides, Neurotoxin substrates or products, respectively. As used herein, the term “isolated” such as an isolated antibody refers to a molecule separated from its natural environment by the use of human intervention.

In one aspect of the method of the invention, the method is a fluorescence method.

In another aspect of the method of the invention, the Neurotoxin polypeptide is a BONT/A, BONT/B, BoNT/C1, BONT/D, BONT/E, BoNT/F or BoNT/G polypeptide or a Tetanus (TeNT) Neurotoxin polypeptide, as defined in detail elsewhere herein.

In a further aspect of the method of the invention, the Neurotoxin substrate is VAMP/Synaptobrevin, SNAP-25 or Syntaxin.

In the following, the corresponding accession number of the respective Neurotoxin substrate which can be used in the method of the invention is indicated: human SNAP-25 P60880, human Syntaxin-1A Q16623, Syntaxin-1B P61266, Syntaxin-2 P32856, Syntaxin-3 Q13277, Syntaxin-4 Q12846, Syntaxin-5 Q13190, Syntaxin-6 043752, Syntaxin-7 O15400, Syntaxin-8 Q9UNK0, Syntaxin-10 060499, Syntaxin-11 075558, Syntaxin-12 Q86Y82, Syntaxin-16 O14662, Syntaxin-17 P56962, Syntaxin-18 Q9P2W9, Syntaxin-19 Q8N4C7; human Synaptobrevin-1 P23763, Synaptobrevin-2 P63027, Synaptobrevin-3 Q15836; human synaptotagmin: Synaptotagmin-1 P21579, Synaptotagmin-2 Q8N910, Synaptotagmin-3 Q9BQG1, Synaptotagmin-4 Q9H2B2, Synaptotagmin-5 000445, Synaptotagmin-6 Q5T7P8, Synaptotagmin-8 Q8NBV8, Synaptotagmin-9 Q86SS6, Synaptotagmin-10 Q6XYQ8, Synaptotagmin-11 Q9BT88, Synaptotagmin-12 Q8IV01, Synaptotagmin-13 Q7L8C5, Synaptotagmin-14 Q8NB59, Synaptotagmin-15 Q9BQS2, Synaptotagmin-16 Q17RD7, Synaptotagmin-17 Q9BSW7, human vesicle associated membrane proteins (VAMPs): Vesicle-associated membrane protein 1 P23763, Vesicle-associated membrane protein 2 P63027, Vesicle-associated membrane protein 3 Q15836, Vesicle-associated membrane protein 4 075379, Vesicle-associated membrane protein 5 095183, Vesicle-associated membrane protein 7 P51809, Vesicle-associated membrane protein 8 Q9BV40; of synaptic vesicle glycoproteins (SV2): Synaptic vesicle glycoprotein 2A Q7L0J3, Synaptic vesicle glycoprotein 2B Q7L112, Synaptic vesicle glycoprotein 2C.

In another aspect of the invention, the cells are neuronal cells or neuronal differentiated cells selected from the group consisting of: primary neuronal cells, tumor cells which are capable of differentiating to neuronal cells such as neuroblastoma cells or cell lines as defined elsewhere herein, P19 cells or induced pluripotent stem cell (iPS)-derived neurons, preferably human induced pluripotent stem cell (iPS)-derived neurons.

In a further aspect of the method of the invention, fixing the cells is carried out by the addition of a fixation agent selected from the group consisting of: methanol, ethanol, acetone, formaldehyde or mixtures thereof. Preferably, fixing the cells is carried out by addition of ice-cold methanol (−20° C.) and incubation for about 20 minutes at −20° C.

In one aspect of the method of the invention, the first capture antibody specifically binding to the non-cleaved and Neurotoxin-cleaved substrate allows for the determination of the total amount of the Neurotoxin substrate in the cells. Suitable binding regions and epitopes of the first capture antibody within the respective Neurotoxin substrate(s) have been defined elsewhere herein.

In specific aspects of the method of the invention, the first capture antibody specifically binding to the non-cleaved and Neurotoxin-cleaved substrate is the rabbit polyclonal anti-SNAP-25 antibody S9684, the rabbit polyclonal anit-SNAP25 antibody PA5-19708 (Pierce Antibodies), or the rabbit polyclonal anti-SNAP25 antibody PA5-19701 (Pierce Antibodies).

In further aspects of the method of the invention, the second capture antibody is the mouse monoclonal antibody 20-2-5, 5-10-5, 1-10-4, 16-5-4, 6-3-8, 18-3-3, or 14-12-1 of the invention, or the mouse monoclonal antibody clone MC-6053 (R&D Systems). Preferably, the second capture antibody is the mouse monoclonal antibody 20-2-5. The corresponding sequences of the variable regions and the CDRs of the mouse monoclonal antibodies of the invention have been described elsewhere herein.

In specific aspects of the method of the invention, the first and/or second capture antibody is/are immobilized. For example, said first and/or second capture antibody is/are linked to a solid phase support. As used herein, the term “solid-phase support” is synonymous with “solid phase” and refers to any matrix that can be used for immobilizing a first and/or second capture antibody disclosed in the present specification. Non-limiting examples of solid phase supports include, e.g., a tube; a plate; a column; pins or “dipsticks”; a magnetic particle, a bead or other spherical or fibrous chromatographic media, such as, e.g., agarose, sepharose, silica and plastic; and sheets or membranes, such as, e.g., nitrocellulose and polyvinylidene fluoride (PVDF). The solid phase support can be constructed using a wide variety of materials such as, e.g., glass, carbon, polystyrene, polyvinylchloride, polypropylene, polyethylene, dextran, nylon, diazocellulose, or starch. The solid phase support selected can have a physical property that renders it readily separable from soluble or unbound material and generally allows unbound materials, such as, e.g., excess reagents, reaction by-products, or solvents, to be separated or otherwise removed (by, e.g., washing, filtration, centrifugation, etc.) from solid phase support-bound assay component. Non-limiting examples of how to make and use a solid phase supports are described in, e.g., Molecular Cloning, A Laboratory Manual, supra, (2001); and Current Protocols in Molecular Biology, supra, (2004), each of which is hereby incorporated by reference in its entirety. In one aspect, the first and/or second capture antibody is conjugated to beads. It is envisaged that the antibody-bead conjugates are small enough to be able to enter the cells through the pores caused by the permeabilization of said cells.

In specific aspects of the method of the invention, the first detection antibody is an alkaline phosphatase(AP)-conjugated antibody, a horseradish-peroxidase (HRP)-conjugated antibody or an antibody conjugated to a fluorescence dye.

In further specific aspects of the method of the invention, the second detection antibody is an alkaline phosphatase (AP)-conjugated antibody, a horseradish-peroxidase (HRP)-conjugated antibody, a glucose oxidase-conjugated antibody, a tyrosinase-conjugated antibody or a β-Galactosidase-conjugated antibody.

Preferably, the alkaline phosphatase (AP)-conjugated antibody is used as a first detection antibody for the measurement of the amount of the total (non-cleaved and cleaved) Neurotoxin substrate, such as total SNAP-25, in the cell; and the horseradish peroxidase (HRP)-conjugated antibody is used as a second detection antibody for the measurement of the amount of the cleaved Neurotoxin substrate, such as BoNT/A cleaved SNAP-25, in the cell.

In certain aspects of the method of the invention, the AP substrate is a 4-methylumbelliferryl phosphate derivative such as 6,8-Difluoro-4-methylumbelliferyl phosphate (DiFMUP), or fluorescein diphosphate (FDP).

In specific aspects of the method of the invention, the HRP substrate is Amplex UltraRed, 10-Acetyl-3,7-Dihydroxyphenoxazine (ADHP) or 3-(4-Hydroxyphenyl) propionic acid (HPPA).

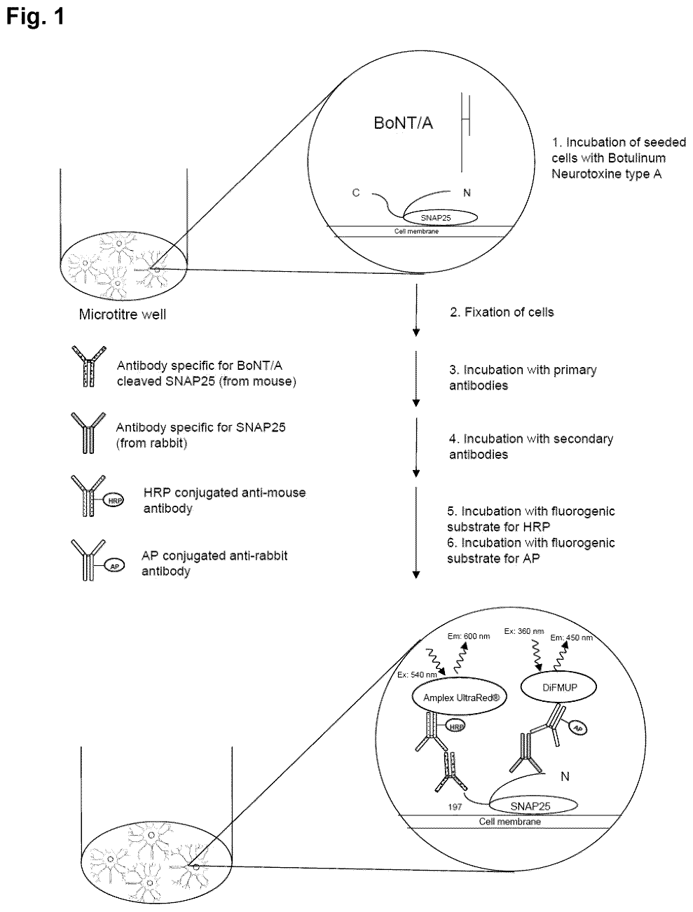

In a more specific aspect of the method of the invention, the method is carried out as illustrated in FIG. 1.

The invention in a further aspect relates to a kit for carrying out the method of the invention comprising:
a) an arrangement of a first capture antibody, a second capture antibody, a first detection antibody and a second detection antibody, wherein said arrangement allows for carrying out the method of the invention;
b) means for calculating the amount of substrate cleaved by said Neurotoxin based on the amounts of the first and second detection complexes determined by the arrangement according to a); and
c) instructions for carrying out said method.

The term “kit” as used herein refers to a collection of the aforementioned means or reagents of the present invention which may or may not be packaged together. The components of the kit may be comprised by separate vials (i.e. as a kit of separate parts) or provided in a single vial. Moreover, itis to be understood that the kit of the present invention is to be used for practicing the methods referred to herein above. In one aspect, it is envisaged that all components are provided in a ready-to-use manner for practicing the method referred to herein. In a further aspect, the kit contains instructions for carrying out the said method. The instructions can be provided by a user manual in paper- or electronic form. For example, the manual may comprise instructions for interpreting the results obtained when carrying out the aforementioned methods using the kit of the present invention.

Finally, the invention relates in another aspect to a method for manufacture of a formulated Neurotoxin product for use in pharmaceutical or cosmetic applications, comprising (i) determining the biological activity of a Neurotoxin product by the method of the invention and (ii) formulating the Neurotoxin product for use in pharmaceutical or cosmetic applications. The Neurotoxin product can be formulated by various techniques dependent on the desired application purposes which are known in the art. For example, the (biologically active) Neurotoxin product can be used in combination with one or more pharmaceutically acceptable carriers as a pharmaceutical composition. The pharmaceutically acceptable carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatine, agar, pectin, acacia, magnesium stearate, stearic acid and the like. Exemplary of liquid carriers are glycerol, phosphate buffered saline solution, water, emulsions, various types of wetting agents, and the like. Suitable carriers comprise those mentioned above and others well known in the art, see, e.g., Remington's Pharmaceutical Sciences, Mack Publishing Company, Easton, Pennsylvania. In an aspect, the pharmaceutical composition can be dissolved in a diluent, prior to administration. The diluent is also selected so as not to affect the biological activity of the Neurotoxin product. Examples of such diluents are distilled water or physiological saline. In addition, the pharmaceutical composition or formulation may also include other carriers or non-toxic, non-therapeutic, non-immunogenic stabilizers and the like. Thus, the formulated Neurotoxin product can be present, in an aspect, in liquid or lyophilized form. In an aspect, it can be present together with glycerol, protein stabilizers (HSA) or non-protein stabilizers such as polyvinyl pyrrolidone (PVP), hyaluronic acid or free amino acids. In an aspect, suitable non-proteinaceous stabilizers are disclosed in WO 2005/007185 or WO 2006/020208. In one aspect, the biological activity determined according to step (i) by the method of the invention corresponds to an Botulinum toxin activity of 25, 50, 75, 100, 125, 150 or 200 U (Mouse LD50 units). The formulated Neurotoxin product may be used for human or animal therapy of various diseases or disorders in a therapeutically effective dose or for cosmetic purposes.

The disease or disorder as referred to herein is selected from the group consisting of voluntary muscle strength, focal dystonia, including cervical, cranial dystonia, and benign essential blepharospasm, hemifacial spasm, and focal spasticity, gastrointestinal disorders, hyperhidrosis, and cosmetic wrinkle correction, Blepharospasm, oromandibular dystonia, jaw opening type, jaw closing type, bruxism, Meige syndrome, lingual dystonia, apraxia of eyelid, opening cervical dystonia, antecollis, retrocollis, laterocollis, torticollis, pharyngeal dystonia, laryngeal dystonia, spasmodic dysphonia/adductor type, spasmodic dysphonia/abductor type, spasmodic dyspnea, limb dystonia, arm dystonia, task specific dystonia, writer's cramp, musician's cramps, golfer's cramp, leg dystonia, thigh adduction, thigh abduction knee flexion, knee extension, ankle flexion, ankle extension, equinovarus, deformity foot dystonia, striatal toe, toe flexion, toe extension, axial dystonia, pisa syndrome, belly dancer dystonia, segmental dystonia, hemidystonia, generalised dystonia, dystonia in lubag, dystonia in corticobasal degeneration, dystonia in lubag, tardive dystonia, dystonia in spinocerebellar ataxia, dystonia in Parkinson's disease, dystonia in Huntington's disease, dystonia in Hallervorden-Spatz disease, dopa-induced dyskinesias/dopa-induced dystonia, tardive dyskinesias/tardive dystonia, paroxysmal dyskinesias/dystonias, kinesiogenic non-kinesiogenic action-induced palatal myoclonus, myoclonus myokymia, rigidity, benign muscle cramps, hereditary chin trembling, paradoxic jaw muscle activity, hemimasticatory spasms, hypertrophic branchial myopathy, maseteric hypertrophy, tibialis anterior hypertrophy, nystagmus, oscillopsia supranuclear gaze palsy, epilepsia, partialis continua, planning of spasmodic torticollis operation, abductor vocal cordparalysis, recalcitant mutational dysphonia, upper oesophageal sphincter dysfunction, vocal fold granuloma, stuttering Gilles de la Tourette syndrome, middle ear myoclonus, protectivelarynxclosure, postlaryngectomy, speechfailure, protective ptosis, entropion sphincter Odii dysfunction, pseudoachalasia, nonachalsia, oesophageal motor disorders, vaginismus, postoperative immobilisation tremor, bladder dysfunction, detrusor sphincter dyssynergia, bladder sphincter spasm, hemifacial spasm, reinnervation dyskinesias, mentalis dimples, stiff person syndrome, tetanus prostate hyperplasia, adipositas, treatment infantile cerebral palsy strabismus, mixed paralytic concomitant, after retinal detachment surgery, after cataract surgery, in aphakia myositic strabismus, myopathic strabismus, dissociated vertical deviation, as an adjunct to strabismus surgery, esotropia, exotropia, achalasia, anal fissures, exocrine gland hyperactivity, Frey syndrome, Crocodile Tears syndrome, hyperhidrosis, axillar palmar plantar rhinorrhea, relative hypersalivation in stroke, in Parkinson's, in amyotrophic lateral sclerosis, spastic conditions, in encephalitis and myelitis autoimmuneprocesses, multiple sclerosis, transverse myelitis, Devic syndrome, viral infections, bacterial infections, parasitic infections, fungal infections, in hereditary spastic paraparesis postapoplectic syndrome hemispheric infarction, brainstem infarction, myelon infarction, in central nervous system trauma, hemispheric lesions, brainstem lesions, myelon lesion, in central nervous system hemorrhage, intracerebral hemorrhage, subarachnoidal hemorrhage, subdural hemorrhage, intraspinal hemorrhage, in neoplasias, hemispheric tumors, brainstem tumors, myelon tumor and vaginism. A cosmetic use is selected from treatment or reduction of wrinkles like crow's feet or GFL, frowning, facial asymmetries.

All references cited in this specification are herewith incorporated by reference with respect to their entire disclosure content and the disclosure content specifically mentioned in this specification.

The figures show:

FIG. 1: Diagram representing the mode of action of the cell-based assay of the invention. Cells susceptible to Neurotoxin intoxication are seeded in multiwell plates, Thereafter, the cells are intoxicated with Neurotoxin polypeptide and after a given intoxication period the cells are fixated. The specific antibody for Neurotoxin-cleaved SNAP-25 and the specific antibody for un-cleaved SNAP-25 bind to the specific binding sites on SNAP-25. Using enzyme-coupled anti-host specific secondary antibodies, these binding events can be used to generate measurable signals which correlate with the concentration of neurotoxin cleaved SNAP-25 and the total amount of SNAP-25 within the well. With increasing BoNT/A concentration the amount of measured cleaved SNAP-25 increases resulting in a gain of signal.

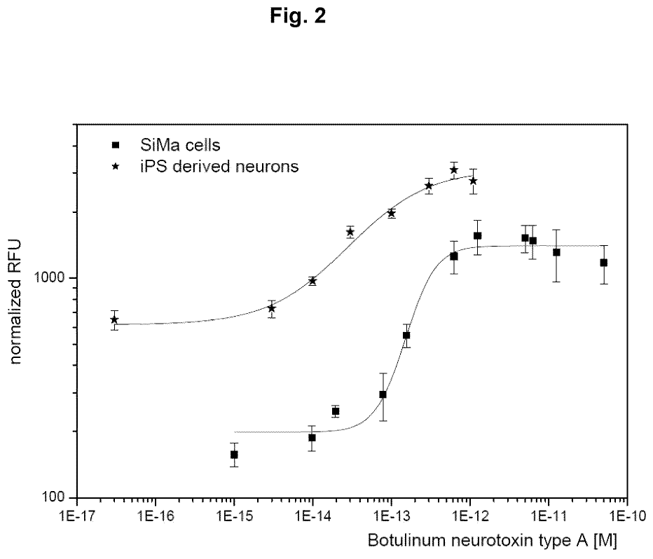

FIG. 2: The two graphs represent the resulting BoNT/A calibration curves for iPS-derived neurons and SiMa cells according to Example 2. They show the dependency between respectively the concentration and activity of BoNT/A and the determined fluorescence signal (RFU) for the HRP substrate and the content of BoNT/A-cleaved SNAP-25 normalized to the total amount of SNAP-25 within the well. Upon increasing concentration and activity, respectively, of BoNT/A, more SNAP-25 is converted by the Neurotoxin, resulting in an increase in the content of cleaved SNAP-25.

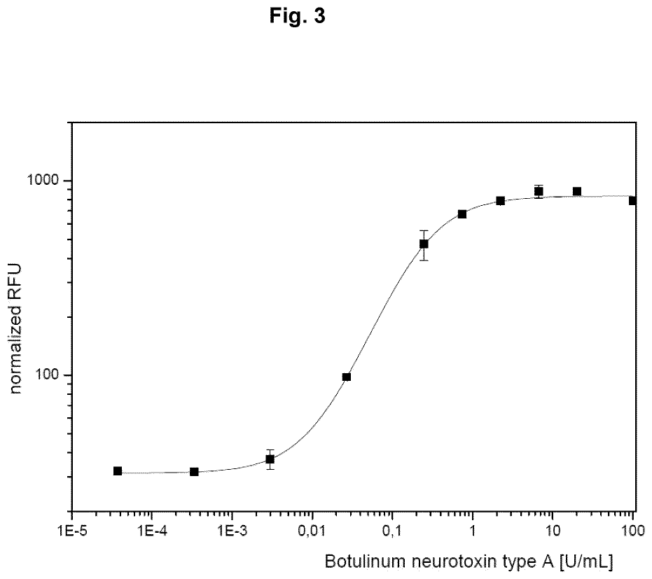

FIG. 3: The graph represents the resulting BoNT/A calibration curve for iPS derived neurons according to Example 4. It shows the dependency between respectively the concentration and activity of BoNT/A and the determined fluorescence signal (RFU) for the HRP substrate and the content of BoNT/A-cleaved SNAP-25 and the content of BoNT/A-cleaved SNAP-25 normalized to the total amount of SNAP-25 within the well. Upon increasing concentration and activity, respectively, of BoNT/A, more SNAP-25 is converted by the Neurotoxin, resulting in an increase in the content of cleaved SNAP-25.

The invention will now be illustrated by the following examples which shall, however, not be construed as limiting the scope of the present invention.

### EXAMPLE 1: GENERATION OF MONOCLONAL ANTIBODIES SPECIFICALLY BINDING TO THE CLEAVAGE SITE OF THE NEUROTOXIN-CLEAVED SUBSTRATE SNAP-25

Mouse monoclonal antibodies specifically binding to the cleavage site of the Neurotoxin-cleaved substrate SNAP-25 have been generated using the hybridoma standard technique. To this end, Balb/c mice (female, 8 weeks) have been immunized with SNAP-25₁₉₀₋₁₉₇ with a Cysteine residue at the N-terminus, “C-TRIDEANQ” (SEQ ID NO: 17). Said N-terminal Cysteine residue is not derived from the SNAP-25 amino acid sequence but has been introduced for linking the SNAP-25₁₉₀₋₁₉₇ peptide (SEQ ID NO: 74) to the keyhole limpet hemocyanin (KLH). Hybridoma cells have been obtained by the fusion of mouse spleen cells with the myeloma cell line SP2/0-Ag14 (SP2/0) purchased from the German Collection of Microorganisms and Cell Culture (DSMZ GmbH, Braunschweig, ACC 146); see also Hemmerlein et al., Molecular Cancer 2006, 5, 41. Antibodies specifically binding to the cleavage site of the Neurotoxin-cleaved substrate SNAP-25 were screened in ELISA. The obtained clones have been selected with respect to their specificity and affinity to BoNT/A-cleaved SNAP-25. As a negative control, the clones have been tested for their non-binding to non-cleaved SNAP-25₂₀₆. As a result, the mouse monoclonal antibodies 20-2-5, 5-10-5, 1-10-4, 16-5-4, 6-3-8, 18-3-3, and 14-12-1 were found to be highly specific for BoNT/A-cleaved SNAP-25₁₉₇, with no detectable cross-reactivity to SNAP25₂₀₆ in ELISA and Western blots. Isotyping of said monoclonal antibodies has been carried out using the mouse monoclonal antibody isotyping test kit (Serotec). As a result it has been found that mAb 20-2-5, 14-12-1, 6-3-8, and 5-10-5 are IgG1 antibodies, whereas mAb 18-3-3, 16-5-4, and 1-10-4 are IgG2a antibodies.

The corresponding amino acid sequences of the VH and VL chains and the corresponding CDR (complementarity determining region) sequences of the mentioned mouse monoclonal antibodies are indicated in the sequence listing.

### EXAMPLE 2: DOUBLE-FLUORESCENCE-CB-BONT/A ACTIVITY ELISA

### Fixation of Cells

1. Remove the media/toxin solution. Add 100 μl/well ice-cold methanol (−20° C.) and incubate for 20 min at −20° C.
   Note: Perform all subsequent steps at room temperature.

### After Cell Fixation:

1. Remove the methanol solution and add 100 μl/well PBS buffer. For longer storage (>1 day) one should add 300 μl//well PBS buffer and seal the plates with parafilm. The plates should be stored in the refrigerator.
2. Remove the PBS buffer and wash the cells 3 times with 200 μl/well of wash buffer. Each step should be performed for 1 minute with gentle shaking.
3. Remove the wash buffer and add 100 μl/well of quenching buffer and incubate for 20 minutes with gentle shaking.
4. Remove the quenching buffer and wash the cells once with 300 μl/well of wash buffer for 5 minutes under gentle shaking.
5. Remove the wash buffer, and add 200 μl/well of blocking buffer and incubate for 1 hour with gentle shaking.
6. Remove the blocking buffer, and add 100 μl/well permeabilization buffer and incubate for 15 minutes with gentle shaking.
7. Remove the permeabilization buffer and wash the cells once with 300 μl/well of PBS buffer. This step should be performed for 1 minute with gentle shaking.
8. Remove the PBS buffer and add 100 μl of the primary antibody mixture (antibody dilution in blocking buffer) to each well. Incubate overnight (16-18 h) with gentle shaking. The cells are simultaneously incubated with two primary antibodies: a mouse antibody specific for the BoNT/A-cleaved SNAP-25 and a polyclonal rabbit antibody that recognizes SNAP-25 (antibody for determining the total amount of SNAP-25 for normalization).
9. Remove the primary antibody mixture and wash the cells 4 times with 200 μl of wash buffer. Each step should be performed for 5 minutes with gentle shaking.
10. Remove the wash buffer, and add 100 μl of the secondary antibody mixture: HRP-conjugated anti-mouse and AP-conjugated anti-rabbit secondary antibodies (antibody dilution in blocking buffer) to each well and incubate for 2.5-3 hours with gentle shaking.
11. Remove the secondary antibody mixture and wash the cells 5 times with 200 μl/well of wash buffer, followed by 1 washing step with 300 μl/well of HEPES buffer. Each wash step should be performed for 5 minutes with gentle shaking.
12. Remove the PBS buffer from the plate and add 75 μl of a fluorogenic substrate for horseradish-peroxidase (HRP substrate) to each well. Incubate for 50 minutes with gentle shaking. Protect the plates from direct light.
13. Add 75 μl of a fluorogenic substrate for alkaline phosphatase (AP substrate) to each well and incubate for an additional 50 minutes at with gentle shaking. Protect the plates from direct light.
14. Read the plates using a fluorescence plate reader:
   excitation at 540 nm; emission at 600
   nm.excitation at 360 nm; emission at 450 nm.
15. Calculation
   For normalization, the RFU value for cleaved SNAP-25 (fluorescence at 600 nm) is normalized to RFU of total SNAP-25 (450 nm) in each well. For better illustration of RFUs in a diagram all values are multiplied with a factor 1000 using the following equation:

$$RFU ( 600 nm ) RFU ( 450 nm ) \times 1000$$

Subsequently the resulting RFU values are averaged for each standard or sample.

### Reagent Preparation

### Wash Buffer:

0.1% Triton X-100 in 10 mM PBS buffer (pH 7.4)

### PBS Buffer (10 mM):

Phosphate buffered saline (Sigma, #P5368) (pH 7.4)

### Quenching Buffer:

0.6% H₂O₂ in 10 mM PBS buffer (pH 7.4)

### Blocking Buffer:

2% BSA in 10 mM PBS buffer (pH 7.4)+0.05% Triton X-100

### Permeabilization Buffer:

0.5% Triton X-100 in 10 mM PBS buffer

### HEPES Buffer:

50 mM HEPES (pH 7.4)

### HRP Substrate:

50 mM HEPES (pH 7.4)
0.007% H₂O₂
150 pM Amplex UltraRed

### AP Substrate:

25 mM Diethanolamine (pH 9.8)
2 mM MgCl₂
100 μl M DiFMUP

### EXAMPLE 3: ILLUSTRATION OF BONT/A CALIBRATION CURVES IN THE CBA-ELISA ACCORDING TO EXAMPLE 2 OF THE PRESENT INVENTION

Cell culture and intoxication with BoNT/A of parental SiMa cells has been carried out according to the provider's manual. Similarly, cell culture and intoxication with BoNT/A of human induced pluripotent stem (iPS)cell-derived neurons (Cellular Dynamics) has been carried out according to the protocol by the manufacturer.

The ELISA has been carried out according to Example 2. As first capture antibody specifically binding to the non-cleaved and BoNT/A-cleaved SNAP-25, the rabbit polyclonal anti-SNAP-25 antibody S9684 (Sigma) has been used. This antibody allows for the detection of the total amount of SNAP-25 within the cells. As a second capture antibody specifically binding to the cleavage site of the BoNT/A-cleaved SNAP-25, the monoclonal antibody clone 20-2-5 of the invention (see Example 1) has been utilized.

The two graphs in FIG. 2 show the obtained BoNT/A calibration curves. They demonstrate the dependency between respectively the concentration and activity of BoNT/A and the determined fluorescence signal (RFU) for the HRP substrate and the content of BoNT/A-cleaved SNAP-25 (RFU values are not blank-corrected in order to illustrate the errors of the single BoNT/A standards). Upon increasing concentration and activity, respectively, of BoNT/A, more SNAP-25 is converted by the Neurotoxin resulting in an increase in the content of cleaved SNAP-25. The dependency of the signal of the BoNT/A concentration/activity of BoNT/A is illustrated by using a 4-parameter equation.

### EXAMPLE 4: DOUBLE-FLUORESCENCE-CB-BONT/A ACTIVITY ELISA

### Fixation of Cells

1. Remove the media/toxin solution. Add 100 μl/well ice-cold methanol (−20° C.) and incubate for 20 min at −20° C.
   Note: Perform all subsequent steps at room temperature.

### After Cell Fixation:

1. Remove the methanol solution and add 100 μl/well PBS buffer. For longer storage (>1 day) one should add 300 μl//well PBS buffer and seal the plates with parafilm. The plates should be stored in the refrigerator.
2. Remove the PBS buffer and wash the cells 3 times with 200 μl/well of PBS buffer. Each step should be performed for 1 minute with gentle shaking.
3. Remove the PBS buffer and add 100 μl/well of quenching buffer and incubate for 20 minutes with gentle shaking.
4. Remove the quenching buffer and wash the cells once with 300 μl/well of PBS buffer for 3 minutes under gentle shaking.
5. Remove the PBS buffer, and add 200 μl/well of blocking buffer and incubate for 1 hour with gentle shaking.
6. Remove the blocking buffer and add 100 μl of the primary antibody mixture (antibody dilution in blocking buffer) to each well. Incubate overnight (16-18 h) with gentle shaking. The cells are simultaneously incubated with two primary antibodies: a mouse antibody specific for the BoNT/A-cleaved SNAP-25 and a polyclonal rabbit antibody that recognizes SNAP-25 (antibody for determining the total amount of SNAP-25 for normalization).
7. Remove the primary antibody mixture and wash the cells 4 times with 200 μl of PBS buffer. Each step should be performed for 3 minutes with gentle shaking.
8. Remove the PBS buffer, and add 100 μl of the secondary antibody mixture: HRP-conjugated anti-mouse and AP-conjugated anti-rabbit secondary antibodies (antibody dilution in blocking buffer) to each well and incubate for 2.5-3 hours with gentle shaking.
9. Remove the secondary antibody mixture and wash the cells 5 times with 200 μl/well of PBS buffer, followed by 1 washing step with 300 μl/well of HEPES buffer. Each wash step should be performed for 3 minutes with gentle shaking.
10. Remove the HEPES buffer from the plate and add 75 μl of a fluorogenic substrate for horseradish-peroxidase (HRP substrate) to each well. Incubate for 50 minutes with gentle shaking. Protect the plates from direct light.
11. Add 75 μl of a fluorogenic substrate for alkaline phosphatase (AP substrate) to each well and incubate for an additional 50 minutes at with gentle shaking. Protect the plates from direct light.
12. Read the plates using a fluorescence plate reader:
   excitation at 540 nm; emission at 600 nm.
   excitation at 360 nm; emission at 450 nm.
13. Calculation
   For normalization, the RFU value for cleaved SNAP-25 (fluorescence at 600 nm) is normalized to RFU of total SNAP-25 (450 nm) in each well. For better illustration of RFUs in a diagram all values are multiplied with a factor 1000 using the following equation:

$$RFU ( 600 nm ) RFU ( 450 nm ) \times 1000$$

Subsequently the resulting RFU values are averaged for each standard or sample.

### Reagent Preparation

### PBS Buffer (10 mM):

Phosphate buffered saline (Sigma, #P5368) (pH 7.4)

### Quenching Buffer:

1.6% H₂O₂ in 10 mM PBS buffer (pH 7.4)

### Blocking Buffer:

2% BSA in 10 mM PBS buffer (pH 7.4)+0.05% Triton X-100

### HEPES Buffer:

50 mM HEPES (pH 7.4)

### HRP Substrate:

50 mM HEPES (pH 7.4)
1.7 7% H₂O₂
150 pM Amplex UltraRed

### AP Substrate:

25 mM Diethanolamine (pH 9.8)
2 mM MgCl₂
100 μl M DiFMUP

### EXAMPLE 5: ILLUSTRATION OF BONT/A CALIBRATION CURVES IN THE CBA-ELISA ACCORDING TO EXAMPLE 4 OF THE PRESENT INVENTION

Cell culture and intoxication with BoNT/A of human induced pluripotent stem (iPS) cell-derived neurons (Cellular Dynamics) has been carried out according to the protocol by the manufacturer.

The ELISA has been carried out according to Example 4. As first capture antibody specifically binding to the non-cleaved and BoNT/A-cleaved SNAP-25, the rabbit polyclonal anti-SNAP-25 antibody S9684 (Sigma) has been used. This antibody allows for the detection of the total amount of SNAP-25 within the cells. As a second capture antibody specifically binding to the cleavage site of the BoNT/A-cleaved SNAP-25, the monoclonal antibody clone 20-2-5 of the invention (see Example 1) has been utilized.

The graph shown in FIG. 3 represents the obtained BoNT/A calibration curve. It shows the dependency between respectively the concentration and activity of BoNT/A and the determined fluorescence signal (RFU) for the HRP substrate and the content of BoNT/A-cleaved SNAP-25. Upon increasing concentration and activity, respectively, of BoNT/A, more SNAP-25 is converted by the Neurotoxin resulting in an increase in the content of cleaved SNAP-25. The dependency of the signal of the BoNT/A concentration/activity of BoNT/A is illustrated by using a 4-parameter equation.

1. A kit for directly determining the biological activity of a Neurotoxin polypeptide in cells, comprising
a) an arrangement of:
i) a first capture antibody which specifically binds to a non-cleaved Neurotoxin substrate SNAP-25 and a Neurotoxin-cleaved substrate SNAP-25;
ii) a second capture antibody which specifically binds to the cleavage site of the Neurotoxin-cleaved substrate SNAP-25 and does not bind non-cleaved Neurotoxin substrate SNAP-25, under conditions which allow for binding of the first and second capture antibodies to the substrates, and wherein the second capture antibody is a mouse monoclonal antibody clone 20-2-5 comprising a complementarity determining region (CDR) heavy chain variable region 1 having the sequence of SEQ ID NO:20; a CDR heavy chain variable region 2 having the sequence of SEQ ID NO:21; a CDR heavy chain variable region 3 having the sequence of SEQ ID NO:22; a CDR light chain variable region 1 having the sequence of SEQ ID NO:23; a CDR light chain variable region 2 having the sequence of SEQ ID NO:24; and a CDR light chain variable region 3 having the sequence of SEQ ID NO:25, or a mouse monoclonal antibody MC-6053;
iii) a first detection antibody which specifically binds to the first capture antibody under conditions which allow for binding of the first detection antibody to the first capture antibody, thus forming first detection complexes; and
iv) a second detection antibody which specifically binds to the second capture antibody under conditions which allow for binding of the second detection antibody to the second capture antibody, thus forming second detection complexes;
b) means for calculating the amount of SNAP-25 substrate cleaved by the Neurotoxin based on the amounts of the first and second detection complexes determined by the arrangement according to a); and
c) instructions for directly determining the biological activity of a Neurotoxin polypeptide in cells.

2. The kit of claim 1, wherein the Neurotoxin polypeptide is Clostridium botulinum toxin serotype A (BoNT/A), Clostridium botulinum toxin serotype C1 (BoNT/C1), or Clostridium botulinum toxin serotype E (BoNT/E).

3. The kit of claim 1, wherein the cells susceptible to Neurotoxin intoxication are neuronal cells or neuronal differentiated cells selected from the group consisting of primary neuronal cells, tumor cells which are capable of differentiating to neuronal cells, neuroblastoma cells, P19 cells and induced pluripotent stem (iPS) cell-derived neurons.

4. The kit of claim 1, wherein the first capture antibody specifically binds to the non-cleaved Neurotoxin substrate SNAP-25 and the Neurotoxin-cleaved substrate SNAP-25 and allows for the determination of the total amount of the Neurotoxin substrate in the cells.

5. The kit of claim 1, wherein the first capture antibody which specifically binds to the non-cleaved Neurotoxin substrate SNAP-25 and the Neurotoxin-cleaved substrate SNAP-25 is a rabbit polyclonal anti-SNAP-25 antibody S9684, a rabbit polyclonal anit-SNAP25 antibody PA5-19708, or a rabbit polyclonal anti-SNAP25 antibody PA5-19701.

6. The kit of claim 1, wherein the second capture antibody is a mouse monoclonal antibody clone 20-2-5 or a mouse monoclonal antibody MC-6053.

7. The kit of claim 1, wherein the first and/or second capture antibody is immobilized.

8. The kit of claim 1, wherein the first detection antibody is an alkaline phosphatase (AP)-conjugated antibody, a horseradish-peroxidase (HRP)-conjugated antibody or an antibody conjugated to a fluorescent dye.

9. The kit of claim 1, wherein a substrate for the HRP-conjugated antibody is selected from the group consisting of Amplex UltraRed, 10-Acetyl-3,7-Dihydroxyphenoxazine (ADHP) and 3-(4-Hydroxyphenyl) propionic acid (HPPA).

10. The kit of claim 1, wherein a substrate for the AP-conjugated antibody is selected from the group consisting of 4-methylumbelliferryl phosphate derivative selected from 6,8-Difluoro-4-methylumbelliferyl phosphate (DiFMUP) and fluorescein diphosphate (FDP).

11. The kit of claim 1, wherein the second detection antibody is an alkaline phosphatase (AP)-conjugated antibody, a horseradish-peroxidase (HRP)-conjugated antibody, a glucose oxidase-conjugated antibody, a tyrosinase-conjugated antibody or a β-Galactosidase-conjugated antibody.

12. The kit of claim 1, wherein a substrate for the HRP-conjugated antibody is selected from the group consisting of Amplex UltraRed, 10-Acetyl-3,7-Dihydroxyphenoxazine (ADHP) and 3-(4-Hydroxyphenyl) propionic acid (HPPA).

13. The kit of claim 1, wherein a substrate for the AP-conjugated antibody is selected from the group consisting of 4-methylumbelliferryl phosphate derivative selected from 6,8-Difluoro-4-methylumbelliferyl phosphate (DiFMUP) and fluorescein diphosphate (FDP).


SEQUENCE LISTING (77 sequences)

SEQ ID NO: 1 - DNA, 3891 bp, Clostridium botulinum
  source 1..3891: mol_type = other DNA
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3876 bp, Clostridium botulinum
  source 1..3876: mol_type = other DNA
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaagctccaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaataa

SEQ ID NO: 4 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 5 - DNA, 3843 bp, Clostridium botulinum
  source 1..3843: mol_type = other DNA
atgccaataacaattaacaactttaattattcagatcctgttgataataaaaatattttatatttagatactcatttaaatacattagctaatgagcctgaaaaagcctttcgcattatagggaatatatgggtaatacccgatagattttcaagagattctaatccaaatttaaataaacctcctcgagttacaagccctaaaagtggttattatgatcctaattatttgagtactgattctgaaaaagatacatttttaaaagaaattataaagttatttaaaagaattaactctagagaaataggagaagaattaatatatagacttgcaacagacataccctttcctgggaataacaatactccaattaatacttttgattttgatgtagattttaacagtgttgatgttaaaactagacaaggtaacaactgggttaaaactggtagtataaatcctagtgttataataactggacctagagaaaacattatagacccagaaacttctacgtttaaattaactaacaatacttttgcggcacaagaaggatttggtgctttatcaataatttcaatatcacctagatttatgctaacatatagtaatgcaactaataatgtaggagagggtagattttctaagtctgaattttgcatggatccaatactaattttaatgcatgaacttaatcatgcaatgcataatttatatggaatagctataccaaatgatcaaagaatttcatctgtaactagtaatattttttattctcaatataaggtgaaattagagtatgcagaaatatatgcatttggaggtccaactatagaccttattcctaaaagtgcaaggaaatattttgaggaaaaggcattggattattatagatccatagctaaaagacttaatagtataactactgcaaatccttcaagctttaataaatatataggagaatataaacagaaacttattagaaagtatagattcgtagtagaatcttcaggtgaagttgcagtagatcgtaataagtttgctgagttatataaagaacttacacaaatatttacagaatttaactacgctaaaatatataatgtacaaaataggaaaatatatctttcaaatgtatatactccggttacggcaaatatattagacgataatgtttatgatatacaaaatggatttaacatacctaaaagtaatttaaatgtactatttatgggtcaaaatttatctcgaaatccagcattaagaaaagtcaatcctgaaaatatgctttatttatttacaaaattttgccataaagcaatagatggtagatcattatataataaaacattagattgtagagagcttttagttaaaaatactgacttaccctttataggtgatattagtgatatcaaaactgatatatttttaagcaaagatattaatgaagaaactgaagttatagactatccggacaatgtttcagtggatcaagttattctcagtaagaatacctcagaacatggacaactagatttattataccctattattgaaggtgagagtcaagtattaccgggagagaatcaagtcttttatgataatagaactcaaaatgttgattatttgaattcttattattacctagaatctcaaaaactaagtgataatgttgaagattttacttttacgacatcaattgaggaagctttggataatagtggaaaagtatatacttactttcctaaactagctgataaagtaaatacgggtgttcaaggtggtttatttttaatgtgggcaaatgatgtagttgaagattttactacaaatattctaagaaaagatacattagataaaatatcagatgtatcagctattattccctatataggacctgcattaaatataagtaattctgtaagaaggggaaattttactgaagcatttgcagttaccggtgtaactattttattagaagcgtttcaagaatttacaatacctgcacttggtgcatttgtgatttatagtaaggttcaagaaagaaacgagattattaaaactatagataattgtttagaacaaaggattaaaagatggaaagattcatatgaatggatgataggaacgtggttatccaggattactactcaatttaataatataagttatcaaatgtatgattctttaaattatcaggcagatgcaatcaaagataaaatagatttagaatataaaaaatactcaggaagtgataaagaaaatataaaaagtcaagttgaaaatttaaaaaatagtttagatataaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatataattaatgaatatttcaatagtattaatgattcaaaaattttgagcttacaaaacaaaaaaaatgctttagtggatacatcaggatataatgcagaagtgaggctagaaggtgatgttcaagttaatacgatatatacaaatgattttaaattaagtagttcaggagataaaattatagtaaatttaaataataatattttatatagcgctatttatgagaactctagtgttagtttttggattaagatatctaaagatttaactaattctcataatgaatatacaataattaatagtataaaacaaaattctgggtggaaattatgtattaggaatggcaatatagaatggattttacaagatattaatagaaagtataaaagtttaatttttgattatagtgaatcattaagtcatacaggatatacaaataaatggttttttgttactataactaataatataatggggtatatgaaactttatataaatggagaattaaagcagagtgaaagaattgaagatttaaatgaggttaagttagataaaaccatagtatttggaatagatgagaatatagatgagaatcagatgctttggattagagattttaatattttttctaaagaattaagcaatgaagatattaatattgtatatgagggacaaatattaagaaatgttattaaagattattggggaaatcctttgaagtttgatacagaatattatattattaatgataattatatagataggtatatagcacctaaaagtaatatacttgtacttgttcagtatccagatagatctaaattatatactggaaatcctattactattaaatcagtatctgataagaatccttatagtagaattttaaatggagataatataatgtttcatatgttatataatagtgggaaatatatgataataagagatactgatacaatatatgcaatagaaggaagagagtgttcaaaaaattgtgtatatgcattaaaattacagagtaatttaggtaattatggtataggtatatttagtataaaaaatattgtatctcaaaataaatattgtagtcaaattttctctagttttatgaaaaatacaatgcttctagcagatatatataaaccttggagattttcttttgaaaatgcatacacgccagttgcagtaactaattatgagacaaaactattatcaacttcatctttttggaaatttatttctagggatccaggatgggtagagtaa

SEQ ID NO: 6 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYKVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 7 - DNA, 3858 bp, Clostridium botulinum
  source 1..3858: mol_type = other DNA
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcctagacctacttcaaagtatcaaagttattatgatcctagttatttatctactgatgagcaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattgtatggaataaatataccatctgataaaaggattcgtccacaagttagcgagggatttttttctcaagatggacccaacgtacaatttgaggaattatacacatttggaggatcagatgttgaaataatacctcaaattgaaagattacaattaagagaaaaagcattaggtcactataaagatatagcgaaaagacttaataatattaataaaactattccttctagttggagtagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaagcattatctacctgtatttgcaaatatattagatgataatatttatactataataaacggttttaatttaacaactaaaggttttaatatagaaaattcgggtcagaatatagaaaggaatcctgcactacaaaaacttagttcagaaagtgtagtagatttgtttacaaaagtatgtttaagattaacaagaaatagtagagatgattcaacatgtattcaagttaaaaataatacattaccttatgtagctgataaagatagcatttcacaagaaatatttgaaagtcaaattattacagatgagactaatgtagaaaattattcagataatttttcattagatgaatctattttagatgcaaaagtccctactaatcctgaagcagtagatccactgttacccaatgttaatatggaacctttaaatgttccaggtgaagaagaagtattttatgatgatattactaaagatgttgattatttaaactcttattattatttggaagcccaaaaattaagtaataatgttgaaaatattactcttacaacttcagttgaagaagcattaggttatagcaataagatatacacatttttacctagcttagctgaaaaagtgaataaaggtgttcaagcaggtttattcttaaattgggcgaatgaagtagttgaggattttactacaaatattatgaaaaaagatacattggataaaatatcagatgtatcagccataattccatatataggacctgccttaaatataggaaattcagcattaaggggaaactttaagcaagcatttgcaacagctggtgtagcttttttgttagaaggatttccagagtttacaatacctgcactcggtgtatttaccttttatagttctattcaagaaagagagaaaattattaaaactatagaaaattgtttagaacaaagagttaagagatggaaagattcatatcaatggatggtatcaaattggttgtcaagaattactactcgatttaatcatataagttatcaaatgtatgattctttgagttatcaggcagatgcaatcaaagctaaaatagatttagaatataaaaaatactcaggaagtgataaagaaaatataaaaagtcaagttgaaaatttaaaaaatagtttagatgtaaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatatgattaatgaatatttcaatagtattaatgattcaaaaattttgagcttacaaaataaaaaaaatactttgatggatacatcaggatataacgcagaagtgagagtagaaggcaatgttcagcttaatccaatatttccatttgactttaaattaggtagttcaggggatgatagaggtaaagttatagtaacccagaatgaaaatattgtatataatgctatgtatgaaagttttagtattagtttttggattaggataaataaatgggtaagtaatttacctggatatactataattgatagtgttaaaaataactcaggttggagtataggtattattagtaattttttagtgtttactttaaaacaaaatgaaaatagtgaacaagatataaactttagttatgatatatcaaagaatgctgcgggatataataaatggttttttgtaactattactaccaatatgatgggaaatatgatgatttatataaatggaaaattaatagatactataaaagttaaagagttaactggaattaattttagcaaaactataacatttcaaatgaataaaattccaaatactggcttaattacctcagattctgataacatcaatatgtggataagggatttttatatctttgctaaagaattagatgataaagatattaatatattatttaatagcttgcaatatactaatgttgtaaaagattattggggaaatgatttaagatatgataaagaatattacatgattaacgtaaattatatgaatagatatatgtctaaaaaaggcaatggaattgtttttaatacacgtaaaaataataatgacttcaatgaaggatataaaattataataaaaagaattagaggaaatacaaatgatactagagtacgaggagaaaatgtattatattttaatactacaattgataacaaacaatatagtttaggtatgtataaaccttctagaaatctagggactgatttagttccactaggtgcattggatcaaccaatggatgagatacgtaaatatggttcgtttataatacaaccatgcaatacttttgattactatgcatcacaattatttttgtcaagtaatgcaacaacaaataggcttggaatactatcaattggtagttatagtttcaaacttggagatgactattggtttaatcacgaatatttaattcctgttataaaaatagagcattatgcttcattattagaatcaacatcaactcattgggtttttgtacctgcaagtgaataa

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTRFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 9 - DNA, 3756 bp, Clostridium botulinum
  source 1..3756: mol_type = other DNA
atgccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcaccgttttggatcaatagctatagtaacattctcacctgaatattcttttagatttaatgataattgtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttacgaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaaatcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaagctggatacaacaagtgttagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcaattaaaacaataatagaatctaagtataatagttatactttagaggaaaaaaatgagcttacaaataaatatgatattaagcaaatagaaaatgaacttaatcaaaaggtttctatagcaatgaataatatagacaggttcttaactgaaagttctatatcctatttaatgaaaataataaatgaagtaaaaattaataaattaagagaatatgatgagaatgtcaaaacgtatttattgaattatattatacaacatggatcaatcttgggagagagtcagcaagaactaaattctatggtaactgataccctaaataatagtattccttttaagctttcttcttatacagatgataaaattttaatttcatattttaataaattctttaagagaattaaaagtagttcagttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagagataataattcaggatggaaagtatctcttaatcataatgaaataatttggacattcgaagataatcgaggaattaatcaaaaattagcatttaactatggtaacgcaaatggtatttctgattatataaataagtggatttttgtaactataactaatgatagattaggagattctaaactttatattaatggaaatttaatagatcaaaaatcaattttaaatttaggtaatattcatgttagtgacaatatattatttaaaatagttaattgtagttatacaagatatattggtattagatattttaatatttttgataaagaattagatgaaacagaaattcaaactttatatagcaatgaacctaatacaaatattttgaaggatttttggggaaattatttgctttatgacaaagaatactatttattaaatgtgttaaaaccaaataactttattgataggagaaaagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttatttccattatatgctgatacagctaccacaaataaagagaaaacaataaaaatatcatcatctggcaatagatttaatcaagtagtagttatgaattcagtaggaaattgtacaatgaattttaaaaataataatggaaataatattgggttgttaggtttcaaggcagatactgtcgttgctagtacttggtattatacacatatgagagatcatacaaacagcaatggatgtttttggaactttatttctgaagaacatggatggcaagaaaaataa

SEQ ID NO: 10 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 11 - DNA, 3843 bp, Clostridium botulinum
  source 1..3843: mol_type = other DNA
atgccagttgtaataaatagttttaattataatgaccctgttaatgatgagacaattttatacatgcagaaaccatatgaagaaagaagtagaaaatattataaagcttttgagattatgcctaatgtttggataatgcctgagagagatacaataggaactaagcctgatgagtttcaggtgccggattcattaaagaacggaagtagtgcttattatgatcctaattatttaaccactgatgctgaaaaagatagatatttaaaaacaatgataaaattatttaatagaattaatagtaatcctacagggaaagttttgttagaagaagtatcaaatgctagaccatatttaggagatgatgacacgctaattaatgaattccttccagttaatgtaactacaagtgttaatataaaattttcaactgatgttgaaagttcaataatatcgaatcttcttgtattgggagcaggacctgatatatttaaagcttactgtaccccccttgtaaggtttaataagtcagataaattaattgaaccaagtaatcatggttttggatcaattaatatcttgacattttcacctgagtatgaacatatttttaatgatattagtggagggaatcataatagtacagaatcatttattgcagatcctgcaatttcactagctcatgaattgatacatgcactacatggattatacggggctaaggcagttactcataaagagtctctagtagcagagcgaggacctcttatgatagccgaaaagcccataaggctagaagaatttttaacttttggaggtgaggatttaaatatcattcctagtgctatgaaggaaaaaatatataacgatcttttagctaactatgaaaaaatagctactagacttagagaagttaatacggctcctcctggatatgatattaatgaatataaagattattttcaatggaagtatggactagatagaaatgcagatggaagttatactgtgaatagaaataaatttaatgaaatttataaaaaattatatagctttacagagattgacttagcaaataaatttaaagtaaaatgtagaaatacttattttattaaatatggatttgtaaaagttccaaatttgttagatgatgatatttatactgtatcagaggggtttaatataggtaatttagcagtaaacaatcgcggacaaaatataaatttaaatcctaaaattattgattccattccagataaaggtttagtggaaaagattattaaattttgtaagagcattattcctagaaaaggtacgaagcagtcaccgtcactatgcattagagtaaataatagggagttattttttgtagcttcagaaagtagctataatgaaagtgatattaatacacctaaagaaattgacgatacaacaaatctaaataataattatagaaataatttagatgaagttattttagattataatagtgagacaatacctcaaatatcaaatcgaacattaaatacacttgtacaagacaatagttatgtgccaagatatgattctaatggaacaagtgaaatagaggaatatgatgttgttgactttaatgtatttttctatttacatgcacaaaaagtaccagaaggtgaaaccaatataagtttaacttcttcaattgatacagcattattagaagaatccaaagtatatacatttttttcttcagagtttatcgatactatcaataaacctgtaaatgcagcactatttatagattggataagcaaagtaataagagattttaccactgaagctacacaaaaaagtactgttgataagattgcagacatatctttaattgtaccctatgtaggtcttgctttgaatatagttattgaggcagaaaaaggaaattttgaggaggcatttgaattattaggagcgggtattttattagaatttgtgccagagcttacaattcctgtaattttagtgtttacgataaaatcctatatagattcatatgagaataaaaataaagcaattaaagcaataaataattcattaatcgaaagagaagcaaagtggaaagaaatatatagttggatagtatcaaattggcttactagaattaatacgcaatttaataaaagaaaagagcaaatgtatcaggctttacaaaatcaagtagatgcaataaaaacagcaatagaatataaatataataattatacttcagatgagaaaaatagacttgaatctaaatataatatcaataatatagaagaagaattgaataaaaaagtttctttagcaatgaaaaatatagaaagatttatgacagaaagttctatatcttatttaatgaaattaataaatgaagccgaagttggtaaattaaaagaatatgataaacatgttaagagcgatttattagactatattctctaccataaattaatcttaggagagcagacaaaggaattaattgatttggtgactagtactttgaatagtagtattccatttgaactttcttcatatactaatgataaaattctaattatatattttaatagattatataaaaaaattaaagatagttctattttagatatgcgatatgaaaataataaatttatagatatctctggatatggttcaaatataagcattaatggaaacgtatatatttattcaacaaatagaaatcaatttggaatatatagtggtaggcttagtgaagttaatatagctcaaaataatgatattatatacaatagtagatatcaaaattttagtattagtttctgggtaaccattcctaaacactacagacctatgaatcgtaatcgggaatacactataataaattgtatggggaataataattcgggatggaaaatatcacttagaactattagagattgtgaaataatttggactttacaagatacttccggaaataaggaaaaattaatttttaggtatgaagaacttgctagtatatctgattatataaataaatggatttttgtaactattactaataatagattaggcaattctagaatttacatcaatggaaatttaatagttgaaaaatcaatttcgaatttaggtgatattcatgttagtgataatatattatttaaaattgttggttgtgatgatgaaacgtatgttggtataagatattttaaagtttttaatacggaattagataaaacagaaattgagactttatatagtaatgagccagatccaagtatcttaaaagactattggggaaattatttgctatataataaaaaatattatttattcaatttactaagaaaagataagtatattactcggaattcaggcattttaaatattaatcaacaaagaggtgttactggaggcatatctgtttttttgaactataaattatatgaaggagtagaagttattataagaaaaaatgctcctatagatatatctaatacagataattttgttagaaaaaacgatctagcatacattaatgtagtagatcatggtgtagaatatcggttatatgctgatatatcaattacaaaatcagagaaaataataaaattaataagaacatctaatccaaacgatagcttaggtcaaattatagttatggattcaataggaaataattgcacaatgaattttcaaaacaatgatgggagcaatataggattactaggttttcattcagatgatttggttgctagtagttggtattataaccatatacgaagaaacactagcagtaatggatgcttttggagttttatttctaaagagcatggttggaaagaataa

SEQ ID NO: 12 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 13 - DNA, 3894 bp, Clostridium botulinum
  misc_feature 20: note = n is a, c, g, or t
  source 1..3894: mol_type = other DNA
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaaaataccggtaaatctgaacagtgtattattgttaataatgaggatttatttttcatagctaataaagatagtttttcaaaagatttagctaaagcagaaactatagcatataatacacaaaataatactatagaaaataatttttctatagatcagttgattttagataatgatttaagcagtggcatagacttaccaaatgaaaacacagaaccatttacaaattttgacgacatagatatccctgtgtatattaaacaatctgctttaaaaaaaatttttgtggatggagatagcctttttgaatatttacatgctcaaacatttccttctaatatagaaaatctacaactaacgaattcattaaatgatgctttaagaaataataataaagtctatacttttttttctacaaaccttgttgaaaaagctaatacagttgtaggtgcttcactttttgtaaactgggtaaaaggagtaatagatgattttacatctgaatccacacaaaaaagtactatagataaagtttcagatgtatccataattattccctatataggacctgctttgaatgtaggaaatgaaacagctaaagaaaattttaaaaatgcttttgaaataggtggagccgctatcttaatggagtttattccagaacttattgtacctatagttggattttttacattagaatcatatgtaggaaataaagggcatattattatgacgatatccaatgctttaaagaaaagggatcaaaaatggacagatatgtatggtttgatagtatcgcagtggctctcaacggttaatactcaattttatacaataaaagaaagaatgtacaatgctttaaataatcaatcacaagcaatagaaaaaataatagaagatcaatataatagatatagtgaagaagataaaatgaatattaacattgattttaatgatatagattttaaacttaatcaaagtataaatttagcaataaacaatatagatgattttataaaccaatgttctatatcatatctaatgaatagaatgattccattagctgtaaaaaagttaaaagactttgatgataatcttaagagagatttattggagtatatagatacaaatgaactatatttacttgatgaagtaaatattctaaaatcaaaagtaaatagacacctaaaagacagtataccatttgatctttcactatataccaaggacacaattttaatacaagtttttaataattatattagtaatattagtagtaatgctattttaagtttaagttatagaggtgggcgtttaatagattcatctggatatggtgcaactatgaatgtaggttcagatgttatctttaatgatataggaaatggtcaatttaaattaaataattctgaaaatagtaatattacggcacatcaaagtaaattcgttgtatatgatagtatgtttgataattttagcattaacttttgggtaaggactcctaaatataataataatgatatacaaacttatcttcaaaatgagtatacaataattagttgtataaaaaatgactcaggatggaaagtatctattaagggaaatagaataatatggacattaatagatgttaatgcaaaatctaaatcaatatttttcgaatatagtataaaagataatatatcagattatataaataaatggttttccataactattactaatgatagattaggtaacgcaaatatttatataaatggaagtttgaaaaaaagtgaaaaaattttaaacttagatagaattaattctagtaatgatatagacttcaaattaattaattgtacagatactactaaatttgtttggattaaggattttaatatttttggtagagaattaaatgctacagaagtatcttcactatattggattcaatcatctacaaatactttaaaagatttttgggggaatcctttaagatacgatacacaatactatctgtttaatcaaggtatgcaaaatatctatataaagtattttagtaaagcttctatgggggaaactgcaccacgtacaaactttaataatgcagcaataaattatcaaaatttatatcttggtttacgatttattataaaaaaagcatcaaattctcggaatataaataatgataatatagtcagagaaggagattatatatatcttaatattgataatatttctgatgaatcttacagagtatatgttttggtgaattctaaagaaattcaaactcaattatttttagcacccataaatgatgatcctacgttctatgatgtactacaaataaaaaaatattatgaaaaaacaacatataattgtcagatactttgcgaaaaagatactaaaacatttgggctgtttggaattggtaaatttgttaaagattatggatatgtttgggatacctatgataattatttttgcataagtcagtggtatctcagaagaatatctgaaaatataaataaattaaggttgggatgtaattggcaattcattcccgtggatgaaggatggacagaataa

SEQ ID NO: 14 - protein, 1297 aa, Clostridium botulinum
  SITE 7: note = MISC_FEATURE - Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 15 - DNA, 4400 bp, Clostridium tetani
  source 1..4400: mol_type = other DNA
tagcattaaaaaaattagaacctatagtaaataaattaattaatatatagtttttataatttaattatgaataatattcttaagataaaaagtaaatttttaaaaatttaaattttcagtttacaaaaaataacctgattatgttatatgtaattgtaaaaaacatataaaaaatcagaaaaatttaggaggtatattattaatggattaaataataattttttaatttacttttgattaataaatattaaatgtttattttaattaggagatgatacgtatgccaataaccataaataattttagatatagtgatcctgttaataatgatacaattattatgatggagccaccatactgtaagggtctagatatctattataaggctttcaaaataacagatcgtatttggatagtgccggaaaggtatgaatttgggacaaaacctgaagattttaacccaccatcttcattaatagaaggtgcatctgagtattacgatccaaattatttaaggactgattctgataaagatagatttttacaaaccatggtaaaactgtttaacagaattaaaaacaatgtagcaggtgaagccttattagataagataataaatgccataccttaccttggaaattcatattccttactagacaagtttgatacaaactctaattcagtatcttttaatttattagaacaagaccccagtggagcaactacaaaatcagcaatgctgacaaatttaataatatttggacctgggcctgttttaaataaaaatgaggttagaggtattgtattgagggtagataataaaaattacttcccatgtagagatggttttggctcaataatgcaaatggcattttgcccagaatatgtacctacctttgataatgtaatagaaaatattacgtcactcactattggcaaaagcaaatattttcaagatccagcattactattaatgcacgaacttatacatgtactacatggtttatacggaatgcaggtatcaagccatgaaattattccatccaaacaagaaatttatatgcagcatacatatccaataagtgctgaagaactattcacttttggcggacaggatgctaatcttataagtattgatataaaaaacgatttatatgaaaaaactttaaatgattataaagctatagctaacaaacttagtcaagtcactagctgcaatgatcccaacattgatattgatagctacaaacaaatatatcaacaaaaatatcaattcgataaagatagcaatggacaatatattgtaaatgaggataaatttcagatactatataatagcataatgtatggttttacagagattgaattgggaaaaaaatttaatataaaaactagactttcttattttagtatgaatcatgaccctgtaaaaattccaaatttattagatgatacaatttacaatgatacagaaggatttaatatagaaagcaaagatctgaaatctgaatataaaggacaaaatatgagggtaaatacaaatgcttttagaaatgttgatggatcaggcctagtttcaaaacttattggcttatgtaaaaaaattataccaccaacaaatataagagaaaatttatataatagaactgcatcattaacagatttaggaggagaattatgtataaaaattaaaaatgaagatttaacttttatagctgaaaaaaatagcttttcagaagaaccatttcaagatgaaatagttagttataatacaaaaaataaaccattaaattttaattattcgctagataaaattattgtagattataatctacaaagtaaaattacattacctaatgataggacaaccccagttacaaaaggaattccatatgctccagaatataaaagtaatgctgcaagtacaatagaaatacataatattgatgacaatacaatatatcaatatttgtatgctcaaaaatctcctacaactctacaaagaataactatgactaattctgttgatgacgcattaataaattccaccaaaatatattcatattttccatctgtaatcagtaaagttaaccaaggtgcacaaggaattttattcttacagtgggtgagagatataattgatgattttaccaatgaatcttcacaaaaaactactattgataaaatttcagatgtatccactattgttccttatataggacccgcattaaacattgtaaaacaaggctatgagggaaactttataggcgctttagaaactaccggagtggttttattattagaatatattccagaaattactttaccagtaattgcagctttatctatagcagaaagtagcacacaaaaagaaaagataataaaaacaatagataactttttagaaaaaagatatgaaaaatggattgaagtatataaactagtaaaagcaaaatggttaggcacagttaatacgcaattccaaaaaagaagttatcaaatgtatagatctttagaatatcaagtagatgcaataaaaaaaataatagactatgaatataaaatatattcaggacctgataaggaacaaattgccgacgaaattaataatctgaaaaacaaacttgaagaaaaggctaataaagcaatgataaacataaatatatttatgagggaaagttctagatcatttttagttaatcaaatgattaacgaagctaaaaagcagttattagagtttgatactcaaagcaaaaatattttaatgcagtatataaaagcaaattctaaatttataggtataactgaactaaaaaaattagaatcaaaaataaacaaagttttttcaacaccaattccattttcttattctaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgaaggatggacaaatgattaaacagattgatatgttcatgattactctatataaaaaattaaataatataacaatctagctatattatttttgattattttcttaatatatactaataaaataatcaaaatagagcctatcttaaattactgaagggctgtgtcaaaataagattttgacacagcctctactt

SEQ ID NO: 16 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 17 - protein, 9 aa, synthetic construct
  REGION 1..9: note = peptide used for immunization
CTRIDEANQ

SEQ ID NO: 18 - protein, 114 aa, Mus musculus
EVNLVESGGDLVKPGGSLKLSCAVSGFTFSGYAMSWVRQTPEKRLEWVASIATNGSTYYPDTVKGRFTIFRDNARNILYLQMSSLRSEDTAIYYCTRLRGFDNWGQGTTLTVSS

SEQ ID NO: 19 - protein, 112 aa, Mus musculus
DVLMTQIPLSLPVSLGDQASISCRSSQNIVHSNGNTYLEWYLQKSGQSPKLLIYKVFKRLSGVPDRFSGSGSGTDFTLRISRVEAEDLGVYYCFQGSHVPYTFGGGTKLEIK

SEQ ID NO: 20 - protein, 10 aa, Mus musculus
GFTFSGYAMS

SEQ ID NO: 21 - protein, 16 aa, Mus musculus
SIATNGSTYYPDTVKG

SEQ ID NO: 22 - protein, 4 aa, Mus musculus
GFDN

SEQ ID NO: 23 - protein, 16 aa, Mus musculus
RSSQNIVHSNGNTYLE

SEQ ID NO: 24 - protein, 7 aa, Mus musculus
KVFKRLS

SEQ ID NO: 25 - protein, 9 aa, Mus musculus
FQGSHVPYT

SEQ ID NO: 26 - protein, 119 aa, Mus musculus
DVQLQESGPGLVKPSQSLSLTCTVTGFSITSDYAWNWIRQFPGNKLEWMGYISYKYGTRYNPSLKGRISITRDTSKNQFFLQLNSLTTEDTAIYYCARRGTDGYGFAYWGQGTLVTVSA

SEQ ID NO: 27 - protein, 111 aa, Mus musculus
DIVLTQSPASLGVSLGQRATISCRASESVSSSGYSYIHWYQQRPGQPPKLLIFLASNLESGVPARFSGSGSGTDFTLNIHPVEEEDAATYYCQHSRELPLTFGAGTKLELK

SEQ ID NO: 28 - protein, 11 aa, Mus musculus
GFSITSDYAWN

SEQ ID NO: 29 - protein, 16 aa, Mus musculus
YISYKYGTRYNPSLKG

SEQ ID NO: 30 - protein, 9 aa, Mus musculus
GTDGYGFAY

SEQ ID NO: 31 - protein, 15 aa, Mus musculus
RASESVSSSGYSYIH

SEQ ID NO: 32 - protein, 7 aa, Mus musculus
LASNLES

SEQ ID NO: 33 - protein, 9 aa, Mus musculus
QHSRELPLT

SEQ ID NO: 34 - protein, 114 aa, Mus musculus
EVKLVESGGDLVKPGGSLKLSCAVSGFSLNTYAMSWVRQTPEQRLEWVASISTNGSTYHSDSVKGRFTISRYNARNILYLQMSSLRSEDSAIYYCTRLRGFDYWGQGTTLTVSS

SEQ ID NO: 35 - protein, 112 aa, Mus musculus
DILMTQTPLSLSVSLGDQASISCRSSQNIVHSNGNTYLEWYLQKPGQSPKPLIYKLSKRFSGVPDRFSGSGSGTYFTLKISRVEAEDLGVYYCFQGSHVPYTFGGGTKLEIK

SEQ ID NO: 36 - protein, 10 aa, Mus musculus
GFSLNTYAMS

SEQ ID NO: 37 - protein, 16 aa, Mus musculus
SISTNGSTYHSDSVKG

SEQ ID NO: 38 - protein, 8 aa, Mus musculus
TRLRGFDY

SEQ ID NO: 39 - protein, 16 aa, Mus musculus
RSSQNIVHSNGNTYLE

SEQ ID NO: 40 - protein, 7 aa, Mus musculus
KLSKRFS

SEQ ID NO: 41 - protein, 9 aa, Mus musculus
FQGSHVPYT

SEQ ID NO: 42 - protein, 114 aa, Mus musculus
EVKLVESGGDLVKPGGSLKLSCAASGFTFNTYAMSWVRQTPEKRLEWVASISSNGSTYHPESVKGRFTISRDIARNILNLQMSSLRSEDTAIYYCSRLRGFDYWGQGTTLTVSS

SEQ ID NO: 43 - protein, 112 aa, Mus musculus
DVLMTQTPLSLPVSLGDQASISCRSSQSIVHSNGNTYLEWYLQKPGQSPKPLIYKVFKRFSGVPDRFSGSGSGTDFTLKISRVEAEDLGVYYCFQGSHVPYTFGGGTKLEIK

SEQ ID NO: 44 - protein, 10 aa, Mus musculus
GFTFNTYAMS

SEQ ID NO: 45 - protein, 16 aa, Mus musculus
SISSNGSTYHPESVKG

SEQ ID NO: 46 - protein, 8 aa, Mus musculus
SRLRGFDY

SEQ ID NO: 47 - protein, 16 aa, Mus musculus
RSSQSIVHSNGNTYLE

SEQ ID NO: 48 - protein, 7 aa, Mus musculus
KVFKRFS

SEQ ID NO: 49 - protein, 9 aa, Mus musculus
FQGSHVPYT

SEQ ID NO: 50 - protein, 114 aa, Mus musculus
EVKLVEFGGGLVEPRGSLKLSCAASGFTFSTYAMSWVRQTPEKRLEWVASIASNGSTYYPDSVRGRFTISRDNARNILYLQMSSLRSEDTAMYYCSRLRGFDYWGQGSTLTVSS

SEQ ID NO: 51 - protein, 112 aa, Mus musculus
DVLMTQIPLSLPVSLGDQASISCRSSQSIVHSNGNTYLEWYLQKPGQSPSLLIYKVFKRLSGVPDRFSGSGSGTEFTLKINRVEAEDLGVYYCFQGSHVPYTFGGGTKLEIK

SEQ ID NO: 52 - protein, 10 aa, Mus musculus
GFTFSTYAMS

SEQ ID NO: 53 - protein, 16 aa, Mus musculus
SIASNGSTYYPDSVRG

SEQ ID NO: 54 - protein, 8 aa, Mus musculus
SRLRGFDY

SEQ ID NO: 55 - protein, 16 aa, Mus musculus
RSSQSIVHSNGNTYLE

SEQ ID NO: 56 - protein, 7 aa, Mus musculus
KVFKRLS

SEQ ID NO: 57 - protein, 9 aa, Mus musculus
FQGSHVPYT

SEQ ID NO: 58 - protein, 114 aa, Mus musculus
EVKLVESGGGLVKPGGSLKLSCAASGFTFRSYAVSWVRQTPEKRLEWVASISTNGSTYYPDSMKGRFTISRDTARNILYLEMNSLRSEDTAMYYCARLRGFDYWGQGTPLTVSS

SEQ ID NO: 59 - protein, 112 aa, Mus musculus
DVLMTQTPLSLPVSLGDQASISCRSSQSIVHSNGNTYLEWYLQKPGQSPKLLIYKVSKRFSGVPDRFSGSGSGTDFTLKITRVEAEDLGVYYCFQGSHVPYTFGGGTKLEIK

SEQ ID NO: 60 - protein, 10 aa, Mus musculus
GFTFRSYAVS

SEQ ID NO: 61 - protein, 16 aa, Mus musculus
SISTNGSTYYPDSMKG

SEQ ID NO: 62 - protein, 7 aa, Mus musculus
RLRGFDY

SEQ ID NO: 63 - protein, 16 aa, Mus musculus
RSSQSIVHSNGNTYLE

SEQ ID NO: 64 - protein, 7 aa, Mus musculus
KVSKRFS

SEQ ID NO: 65 - protein, 9 aa, Mus musculus
FQGSHVPYT

SEQ ID NO: 66 - protein, 114 aa, Mus musculus
EVKLVESGGDLVKPGGSLKLSCAASGFTLSDYAMSWVRQSPEERLEWVASISTNGSTYYPDSVKGRFTISRDNARNILSLQMSSLRSEDTAIYYCTRLRGFDNWGQGTLLTVSS

SEQ ID NO: 67 - protein, 112 aa, Mus musculus
DVLMTQTPLSLPVSLGDQASISCRSSQNIVHSNGNTYLEWYLQKSGQSPKLLMYKVSKRLSGVPDRFSGRGSGTDFTLKISRVEAEDLGVYYCFQGSHVPYTFGGGTKLEIK

SEQ ID NO: 68 - protein, 10 aa, Mus musculus
GFTLSDYAMS

SEQ ID NO: 69 - protein, 16 aa, Mus musculus
SISTNGSTYYPDSVKG

SEQ ID NO: 70 - protein, 8 aa, Mus musculus
TRLRGFDN

SEQ ID NO: 71 - protein, 16 aa, Mus musculus
RSSQNIVHSNGNTYLE

SEQ ID NO: 72 - protein, 7 aa, Mus musculus
KVSKRLS

SEQ ID NO: 73 - protein, 9 aa, Mus musculus
FQGSHVPYT

SEQ ID NO: 74 - protein, 8 aa, Mus musculus
TRIDEANQ

SEQ ID NO: 75 - protein, 7 aa, Mus musculus
RIDEANQ

SEQ ID NO: 76 - protein, 6 aa, Mus musculus
IDEANQ

SEQ ID NO: 77 - protein, 5 aa, Mus musculus
DEANQ